(19) United States
(12) Patent Application Publication
(10) Pub. No.: US 2019/0099185 A1
(43) Pub. Date: Apr. 4, 2019

(54) MEDICAL CONNECTOR DEVICE

(71) Applicant: MEDICAL CONNECTION TECHNOLOGY - MEDICONNTECH-M.C.T. LTD, Beit-Zera (IL)

(72) Inventors: Shay KAHANA, Beit-Zera (IL); Zvi NITZAN, Moshav Zofit (IL)

(21) Appl. No.: 16/085,866

(22) PCT Filed: Mar. 14, 2017
(86) PCT No.: PCT/IL2017/050325

§ 371 Date: Sep. 17, 2018

Related U.S. Application Data
(60) Provisional application No. 62/309,475, filed on Mar. 17, 2016.
(60) Provisional application No. 62/310,748, filed on Mar. 20, 2016.
(60) Provisional application No. 62/358,072, filed on Jul. 4, 2016.
(60) Provisional application No. 62/453,015, filed on Feb. 1, 2017.

(51) Int. Cl. A61B 17/11; A61F 2/06

(52) U.S. Cl. CPC A61B 17/11; A61B 2017/1132; A61B 2017/1107; A61F 2/064

(57) ABSTRACT

The present invention provides devices for connecting biological structures. The device features a tubular member that includes a tubular body. The tubular member may include a plurality of interwoven lengths configured for reversible radial enlargement and reversible radial contraction of the tubular member. In some aspects the tubular member may include a net design of interconnected cells configured for reversible radial enlargement of the tubular member. The tubular member includes a distal opening, a proximal opening, a central cavity within the tubular member for receiving and retaining a biological structure and a plurality of spaced apart protrusions protruding out from the exterior surface of the body of the device for fixing to the device an end of the biological structure and for fixing a second structure to the device to facilitate connecting the biological structure to the second structure.

This application claims priority to U.S. Provisional Patent Application No. 62/309,475 filed on Mar. 17, 2016 and U.S. Provisional Patent Application No. 62/310,748 filed on Mar. 20, 2016 and U.S. Provisional Patent Application No. 62/358072 filed on Jul. 4, 2016 and U.S. Provisional Patent Application No. 62/453,015 filed on Feb. 1, 2017 and incorporated herein by reference in their entirety.

### FIELD OF THE INVENTION

The present invention relates to connector devices and methods of use thereof. Moreover, the present invention is of connector devices for medical application, such as for joining body tissues.

### BACKGROUND OF THE INVENTION

In many surgical procedures, it is necessary to join blood vessels end to end or end to side. The most common device for holding body tissues together after injury, or during surgery is surgical suture. However, suturing is slow, and is associated with leaking from the gaps between each stitch and damage to the vascular wall.

Alternative methods, or adjuvants to the use of sutures include gluing, stapling, clips, laser welding, rings and stents. These methods may be quicker than suturing, but are not ideal and suffer from problems, such as being insufficiently strong, a need to remove the connecting device, causing occlusion, high cost and resulting in rigidity and a non-compliant anastomosis.

It would therefore be desirable to have a device and a method for joining body tissues, which would be quick to use, provide uniform connection and patency without the problem of narrowing the lumen, or restriction of the flexibility of the body tissues. It would also be advantageous if the method was of low cost and did not require unusual experience or skill. The present invention provides such a device and method.

### SUMMARY

The invention may have several aspects. One aspect is a device for connecting at least one biological structure. The at least one biological structure may be a biological tissue, a blood vessel, a ligament, a tendon, an intestinal structure, a nerve or part thereof. The device may include a longitudinal axis and a radial axis substantially perpendicular to the longitudinal axis. The device may feature a tubular member that includes a plurality of interwoven lengths configured for reversible radial enlargement of the tubular member in response to a pushing force being applied to at least one of the first end or the second end of the tubular member, the pushing force acting in the direction of the longitudinal axis and reversible radial contraction of the tubular member in response to a pulling force being applied to at least one of the first end or the second end of the tubular member, the pulling force acting in the direction of the longitudinal axis. The device may include a distal opening at a distal end of the tubular member and a proximal opening at a proximal end of the tubular member. The distal opening may be oppositely disposed to the proximal opening. The tubular member may include an inner surface and a lumen defining a cavity for receiving and retaining a biological structure. The cavity may be for receiving and retaining one biological structure. The device may include a plurality of spaced apart protrusions protruding out from the exterior surface of the body of the device for fixing to the device an end of a biological structure retained in the device, the end everted over the proximal extremity of the device and for fixing an end of a second structure to the external surface of the device to facilitate contact between the end of the second structure and the everted end of the biological structure, for connecting the biological structure to the second structure.

In various embodiments the plurality of spaced apart protrusions may be adjacent to the proximal extremity of the tubular member of the device and may be positioned and angled to fix the biological structure to the device and the second structure to the device. The plurality of spaced apart protrusions may include a first plurality of protrusions and a second plurality of protrusions, wherein the first plurality of protrusions are configured for fixing the everted biological structure to the device and the second plurality of protrusions are configured for fixing the second structure to the device and for facilitating contact of at least part of the second structure with at least the everted part of the biological structure. The first plurality of protrusions may be configured spaced apart about the circumference of the tubular member in a first at least one line adjacent to the proximal end of the device and the second plurality of protrusions may be configured spaced apart about the circumference of the tubular member in a second at least one line adjacent to the proximal end of the device. The first at least one line of protrusions may be positioned at a distance that is nearer to the proximal end of the tubular member than the second at least one line of protrusions. The distance between the first at least one line of protrusions and the second at least one line of protrusions may facilitate flexibility of the device and the biological structure inserted therein. The first plurality of spaced apart protrusions may be at least three. The second plurality of spaced apart protrusions may be at least three. The plurality of spaced apart protrusions may include spikes or hooks adapted to fix the biological structure and the second structure to the device. The at least one of the plurality of interwoven lengths may be configured to provide at least one protrusion. At least one of the plurality of interwoven lengths may include a length with a hook at an extremity of the length, the hook facilitating at least one protrusion. The at least one of the plurality of interwoven lengths configured to provide the at least one protrusion may be thicker than the plurality of interwoven lengths, which are not configured to provide the at least one protrusion. The plurality of interwoven lengths may be rounded. The plurality of interwoven lengths may be flat. The device may tighten about the biological structure when at least one of the pushing force is eliminated, the pulling force is applied, or the biological structure is displaced. The shape of the device may conform to the shape of the biological structure retained in the cavity. The plurality of interwoven lengths may be configured as a tubular braid. The tubular member may be constructed from at least one material selected from the group consisting of metal, plastic, nitinol, alloys of titanium and nickel, stainless steel, platinum, gold, silver, copper, zinc, silicone, ceramic, polytetrafluoroethylene (PTFE), polyethylene, urethane, nylon, polyester, polypropylene, fabric, gut and tissue graft and combinations thereof.

The device may be for connecting a biological structure, which is a blood vessel to a second structure, which is an artery.

A further aspect is a device for connecting a biological structure, wherein the device features a tubular member with a net-like design. The tubular member may include a longitudinal axis and a radial axis at least substantially perpendicular to the longitudinal axis. The tubular member may include a tubular body featuring a net-like design of interconnected cells configured for reversible radial enlargement of the tubular member. The tubular member may include a distal opening at a distal end of the tubular member and a proximal opening at a proximal end of the tubular member. The tubular member may include a central cavity within the tubular member for receiving and retaining the biological structure. The tubular member may include a plurality of spaced apart protrusions protruding out from the exterior surface of the body of the device. The spaced apart protrusions may be for fixing to the device an end of the biological structure everted over the proximal extremity of the device. The spaced apart protrusions may be for fixing an end of a second structure to the external surface of the device to facilitate contact between the end of the second structure and the everted end of the biological structure for connecting the biological structure to the second structure. In various embodiments of the device for connecting a biological structure featuring a tubular body with a net-like design, the tubular body may not be configured for substantial linear expansion and contraction when a pulling or pushing force is applied longitudinally to at least one of the distal end and the proximal end. The net-like design of interconnected cells may feature an array of long sided, open diamond shaped cells. The proximal opening may be formed at an acute angle to the longitudinal axis of the device. The acute angle may be from about ten degrees to about forty degrees. The proximal opening may be formed at a ninety degrees angle to the longitudinal axis of the device. The distal opening may be formed at a ninety degrees angle to the longitudinal axis of the device. The tubular body may feature a tubular body first part and a tubular body second part. The tubular body first part may include a right circular hollow cylinder of connected open cells in a net-like design, the first part extending longitudinally from the distal opening until the nearest point of the proximal opening in relation to the distal opening. The tubular body may feature a tubular body second part continuous and in line with the tubular body first part and featuring the same longitudinal axis as the tubular body first part wherein the tubular body second part includes an incomplete circular hollow cylinder, the proximal opening carved out from the cylinder body at an acute angle to the longitudinal axis to form an incomplete ring of connected open cells in a net-like design in a radial line with the proximal opening. The maximal length of the proximal opening may be greater than the diameter of the tubular body first part. The maximal length of the proximal opening may be about five times greater than the diameter of the tubular body first part. The proximal opening may provide and form substantially the same angle and size of the proximal opening, to an end or opening of a biological structure or non-biological structure affixed to the proximal opening of the device. The plurality of protrusions may include a plurality of spaced apart protrusions positioned about the proximal opening of the device. The plurality of protrusions may be positioned in the same line about the circumference of the proximal opening. At least one protrusion of the plurality of protrusions may be sized for fixing to the device both a biological structure and a second structure to be joined to the biological structure. The device may be formed by laser cutting. The tubular body may be round. The tubular body may be rectangular. The proximal opening may be oval. The device may be for at least one of end to side and end to end anastomosis. The device may be for connecting the biological structure to the second structure at an acute angle equivalent to the angle of the proximal opening of the device. The device may include a restraining mechanism. The restraining mechanism may include a restraining ring and at least one unidirectional stop member. The restraining mechanism may include a restraining ring for encircling the device, wherein the encircling restraining ring is freely moveable along the longitudinal axis of the external surface of the device and at least one unidirectional stop member fixed on the external surface of the tubular member adjacent to the proximal opening of the device and projecting out from the external surface of the tubular member. The at least one unidirectional stop member may allow the encircling restraining ring to traverse the at least one unidirectional stop member in a longitudinal direction towards the proximal end of the device. The at least one unidirectional stop member may prevent the encircling restraining ring from moving back over the at least one unidirectional stop member in a longitudinal direction towards the distal end of the device.

An additional aspect is a device for connecting a biological structure, the device including a tubular member that includes a distal opening at a distal end of the tubular member, a proximal opening at a proximal end of the tubular member, a central cavity within the tubular member for receiving and retaining a biological structure, a tubular body comprised of a net-like design of interconnected cells configured for reversible radial enlargement of the tubular member and a plurality of spaced apart protrusions protruding out from the exterior surface of the tubular body of the device. The tubular body may feature a first section and a second section. The first section may feature a first longitudinal axis, a radial axis at least substantially perpendicular to the first longitudinal axis, the first longitudinal axis extending from the distal opening until a bend in the tubular body. The second section may feature a second longitudinal axis, a radial axis at least substantially perpendicular to the second longitudinal axis, the second longitudinal axis extending from the bend until the proximal opening, wherein the second longitudinal axis is inclined at an angle of less than one hundred and eighty degrees to the first longitudinal axis and the first section and the second section are joined to form a continuous cavity extending from the distal opening until the proximal opening. The plurality of spaced apart protrusions may be for fixing to the device an end of a biological structure retained in the device, the end everted over the proximal extremity of the device and for fixing the end of a second structure to the external surface of the device to facilitate contact between the second structure and the everted end of the biological structure for connecting the biological structure to the second structure. At least part of the second section of the device may be for being retained in the lumen of the second structure. The plurality of spaced apart protrusions may be configured for fixing tissue surrounding the lumen of the second structure to the external surface of the device.

An additional aspect is a method of connecting a biological structure and a second structure, the method including providing a device, wherein the device is for connecting at least one biological structure. The device may include a longitudinal axis and a radial axis at least substantially perpendicular to the longitudinal axis. The device may feature a tubular member that includes a plurality of interwoven lengths configured for reversible radial enlargement of the tubular member when a pushing force is applied to at least one of the first end or the second end of the tubular member, the pushing force acting in the direction of the longitudinal axis and reversible radial contraction in response to a pulling force being applied to at least one of the first end or the second end of the tubular member, the pulling force acting in the direction of the longitudinal axis. The device may include a tubular body featuring a net-like design of interconnected cells configured for reversible radial enlargement of the tubular member. The device may include a distal opening at a distal end of the tubular member, a proximal opening at a proximal end of the tubular member and a central cavity within the tubular member for receiving and retaining the biological structure. The proximal opening may be formed by cutting at an acute angle to the longitudinal axis of the body of the device. The device may include a plurality of spaced apart protrusions protruding out from the exterior surface of the body of the device for fixing to the device an end of the biological structure retained in the device, the end everted over the proximal extremity of the device and for fixing a second structure to the device to facilitate contact between the end of the second structure and the everted end of the biological structure, for connecting the biological structure to the second structure. The method may include inserting the biological structure, or a part thereof through the distal opening of the device and into the cavity of the device, and stopping insertion of the biological structure when the end of the biological structure protrudes from the proximal end of the tubular member of the device. The method may include everting the protruding end of the biological structure, or part thereof over the proximal opening of the tubular member and fixing to the device the everted end of the biological structure, or part thereof with the plurality of spaced apart protrusions on the exterior surface of the tubular member. The affixed end of the biological structure may adopt the dimensions, shape and angle of the proximal opening. The method may include manipulating the second structure and the tubular member with the everted and fixed end of the biological structure retained in the device, to facilitate at least a portion of the second structure, such as an opening to surround and overlie the everted and fixed end of the biological structure. The method may include fixing to the device the exposed end of the second structure with the plurality of spaced apart protrusions on the exterior surface of the tubular member, such that at least part of the second structure overlies and is in substantially direct contact with at least part of the everted biological structure.

In various embodiments of the method of connecting a biological structure and a second structure, the plurality of spaced apart protrusions may include a plurality of protrusions, wherein the same protrusion is for fixing both the everted biological structure to the device and the second structure to the device. Alternatively, the plurality of spaced apart protrusions may include a first plurality of protrusions and a second plurality of protrusions. The first plurality of protrusions may be configured for fixing the everted biological structure to the device and the second plurality of protrusions may be configured for fixing the second structure to the device. The first plurality of protrusions may be configured spaced apart about the circumference of the tubular member in a first at least one line adjacent to the proximal end of the device and the second plurality of protrusions may be configured spaced apart about the circumference of the tubular member in a second at least one line adjacent to the proximal end of the device. The first at least one line may be nearer to the proximal end of the tubular member than the second at least one line. The method may include pushing at least one end of the tubular member for reversible radial enlargement and pulling at least one end of the tubular member to secure the tubular member about the inserted biological structure.

In various embodiments of the method of connecting a biological structure and a second structure, the manipulating may include pulling the opening and exposed end of the second structure over the tubular member and over the everted and fixed end of the biological structure retained in the device. The manipulating may include inserting the proximal end of the tubular member with the everted and fixed end of the biological structure into the opening of the exposed end of the second structure. The manipulating may include inserting the tubular member with the everted and fixed end of the biological structure retained therein, into the exposed end of the second structure until the exposed end of the second structure is adjacent to the second at least one line of the plurality of protrusions and the second at least one line of protrusions can be adapted to fix the exposed end of the second structure to the external surface of the device.

A further aspect is a method of connecting end to end a first structure and a second structure to a third structure. The first structure and the second structure may be parts of the same structure. The first structure may be one end of a graft, the second structure may be a second end of a graft and the third structure may be an artery. The method includes providing a first device. The device may feature a tubular member that includes a plurality of interwoven lengths configured for reversible radial enlargement of the tubular member when a pushing force is applied to at least one of the first end or the second end of the tubular member, the pushing force acting in the direction of the longitudinal axis and reversible radial contraction in response to a pulling force being applied to at least one of the first end or the second end of the tubular member, the pulling force acting in the direction of the longitudinal axis. The device may include a distal opening at a distal end of the tubular member, a proximal opening at a proximal end of the tubular member and a central cavity within the tubular member for accommodating an inserted biological or non-biological structure. The device may include a plurality of spaced apart protrusions protruding out from the exterior surface of the body of the device for fixing to the device an end of the inserted biological or non-biological structure everted over the proximal extremity of the device and for fixing a second structure to the device, to facilitate contact between the end of the second structure with the everted end of the inserted biological or non-biological structure, for connecting the biological or non-biological structure to the second structure. The method may include inserting a first end of a first structure into the first device, everting an end of the first structure over the proximal end of the first device and fixing the everted end of the first structure to the first device. The method may include providing a second device. The device may feature a tubular member that includes a plurality of interwoven lengths configured for reversible radial enlargement of the tubular member when a pushing force is applied to at least one of the first end or the second end of the tubular member, the pushing force acting in the direction of the longitudinal axis and reversible radial contraction in response to a pulling force being applied to at least one of the first end or the second end of the tubular member, the pulling force acting in the direction of the longitudinal axis. The device may include a distal opening at a distal end of the tubular member, a proximal opening at a proximal end of the tubular member and a central cavity within the tubular member for accommodating a biological, or a non-biological structure. The device may include a plurality of spaced apart protrusions protruding out from the exterior surface of the body of the device for fixing to the device an end of the inserted biological or non-biological structure everted over the proximal extremity of the device and for fixing a second structure to the device to facilitate contact between the end of the second structure with the everted end of the inserted biological or non-biological structure for connecting the biological or non-biological structure to the second structure. The method may include inserting a first end of a second structure into the second device, everting the end of the second structure over the proximal end of the second device and fixing the everted end of the second structure to the second device. The method may include inserting the proximal end of the first device on which the proximal end of the first structure is everted and fixed, into the opening of a first end of a third structure, or pulling the first end of a third structure over the first device with the everted and fixed end of the first structure. The method may include fixing the first end of the third structure with the plurality of spaced apart protrusions on the exterior surface of the first device, such that at least part of the third structure overlies and is in substantially direct contact with at least part of the everted first structure. The method may include inserting the proximal end of the second device on which the proximal end of the second structure is everted and fixed, into the opening of the second end of the third structure, or pulling the second end of the third structure over the second device with the everted and fixed end of the second structure. The method may include fixing the second end of the third structure with the plurality of spaced apart protrusions on the exterior surface of the second device, such that at least part of the third structure overlies and is in substantially direct contact with at least part of the everted second structure.

A still further aspect is a system for replacement of and/or augmenting suturing in a bypass procedure. The system may include two devices, one device may be for connecting a first end of a bypass blood vessel to a first opening in a blocked artery on a first side of the blockage and the second device may be for connecting a second end of the bypass blood vessel to a second opening in the blocked artery on a second side of the blockage. The device may feature a tubular member that includes a plurality of interwoven lengths configured for reversible radial enlargement of the tubular member when a pushing force is applied to at least one of the first end or the second end of the tubular member, the pushing force acting in the direction of the longitudinal axis and reversible radial contraction in response to a pulling force being applied to at least one of the first end or the second end of the tubular member, the pulling force acting in the direction of the longitudinal axis. The device may include a tubular body featuring a net-like design of interconnected cells configured for reversible radial enlargement of the tubular member. The device may include a distal opening at a distal end of the tubular member, a proximal opening at a proximal end of the tubular member and a central cavity within the tubular member for accommodating an inserted biological or non-biological structure. The device may include a plurality of spaced apart protrusions protruding out from the exterior surface of the body of the device for fixing to the device an end of the inserted biological or non-biological structure everted over the proximal extremity of the device and for fixing a second structure to the device, to facilitate contact between the end of the second structure with the everted end of the inserted biological or non-biological structure, for connecting the biological or non-biological structure to the second structure. The device may include a proximal opening cut at an acute angle to the longitudinal axis of the device. The device may include a proximal opening cut at a ninety degrees angle to the longitudinal axis of the device. The device may connect a bypass blood vessel, such as a graft to the opening in the blocked blood vessel, such as an artery, at an angle corresponding to the angle of the proximal opening of the device. The device may connect a bypass blood vessel, such as a graft to the opening in an artery at a ninety degrees angle.

Another aspect is a method of production of a device for connecting a biological structure with another structure. The method may include providing a plurality of lengths of filaments, tubularly braiding the plurality of lengths of filaments to form a tubular braid and forming hooks from at least some of the plurality of lengths of filaments to form two separated lines of spaced apart hooks positioned about the circumference of the tubular braid.

In various embodiments of the method of production, forming a hook may include cutting a filament length to form an end at a predetermined position about the circumference of the tubular braid and hooking the end to protrude away from the body of the tubular braid. The plurality of lengths of filaments may include hook forming filaments and non-hook forming filaments and wherein the hook forming filaments may be thicker than the non-hook forming filaments. The plurality of lengths of filaments may be rounded filaments.

Another aspect is a method of performing a bypass procedure. The method may include providing a first device. The method may include inserting a first end of a graft through the distal opening of the first device and into the cavity of the first device. The method may include stopping insertion of the first end of the graft when the first end of the graft protrudes from the proximal end of the first device. The method may include everting the protruding first end of the graft over the proximal opening of the first device. The method may include fixing to the first device the everted first end of the graft with the plurality of spaced apart protrusions on the exterior surface of the tubular member. The affixed first end may adopt the dimensions, shape and angle of the proximal opening of the first device. The method may include manipulating the first device with the everted first end of the graft, into a first opening of an artery to facilitate fixing tissue surrounding the first opening of the artery to the spaced apart protrusions of the first device and to overlie and be in substantially direct contact with the everted and fixed first end of the graft. The method may include providing a second device. The method may include inserting a second end of a graft through the distal opening of the second device and into the cavity of the second device. The method may include stopping the insertion of the second end of the graft when the second end of the graft protrudes from the proximal end of the second device. The method may include everting the protruding second end of the graft over the proximal opening of the second device. The method may include fixing to the second device the everted second end of the graft with the plurality of spaced apart protrusions on the exterior surface of the second device. The affixed second end may adopt the dimensions, shape and angle of the proximal opening of the second device. The method may include manipulating the second device with the everted second end of the graft, into a second opening of an artery to facilitate fixing tissue surrounding the second opening of the artery to the spaced apart protrusions on the second device and to overlie and be in substantially direct contact with the everted and fixed second end of the graft. Manipulating may include inserting the first device and the second device with the respective everted first end and the second end of the graft into the respective opening of the artery at a minimal depth to facilitate fixing the opening of the artery to the spaced apart protrusions. The method may include applying a restraining mechanism to the first device to prevent the first device from being displaced deeper into the opening of the artery. The method may include applying a restraining mechanism to the second device to prevent the second device from being displaced deeper into the opening of the artery. The method may feature after the manipulating of the first device, moving a restraining ring which encircles the first device, longitudinally in a direction towards the proximal opening of the first device along the external surface of the first device, until the restraining ring traverses the at least one unidirectional stop member affixed to the first device. The method may include after the manipulating of the second device, moving a restraining ring which encircles the second device, longitudinally in a direction towards the proximal opening of the second device along the external surface of the second device, until the restraining ring traverses the at least one unidirectional stop member affixed to the second device.

In various embodiments of the method of performing a bypass procedure, the device may feature a tubular member. The tubular member may include a longitudinal axis and a radial axis at least substantially perpendicular to the longitudinal axis. The tubular member may include a tubular body featuring a net-like design of interconnected cells configured for reversible radial enlargement of the tubular member. The tubular member may include a distal opening at a distal end of the tubular member and a proximal opening at a proximal end of the tubular member. The tubular member may include a central cavity within the tubular member for receiving and retaining the biological structure. The tubular member may include a plurality of spaced apart protrusions protruding out from the exterior surface of the body of the device. The spaced apart protrusions may be for fixing to the device an end of the biological structure everted over the proximal extremity of the device. The spaced apart protrusions may be for fixing a second structure to the device to facilitate contact between the end of the second structure and the everted end of the biological structure for connecting the biological structure to the second structure. The first device and the second device may include a proximal opening formed at an acute angle to the longitudinal axis of the device. The device may include a restraining mechanism.

An additional aspect is a method of production of a device with a net-like design for connecting a biological structure to another structure. The method may include providing a tubular member. The method may include cutting the body of the tubular member into a net design of interconnected open cells. The method may include cutting an end of the tubular member at an acute angle to the longitudinal axis to form an angled proximal opening. The method may include cutting the body of the tubular member to form a plurality of hooking protrusions positioned about the proximal opening of the tubular member. The cutting may be done with a laser.

### BRIEF DESCRIPTION OF THE DRAWINGS

The various features of the invention will best be appreciated by simultaneous reference to the description which follows and the accompanying drawings, which are not drawn to scale and in which:

FIG. 1a shows a schematic view of an exemplary connector device according to an aspect of the present invention;

FIG. 1b shows a schematic view of an exemplary connector device according to an aspect of the present invention;

FIG. 1c shows a schematic view of an exemplary connector device according to an aspect of the present invention;

FIG. 2a shows a schematic view of an exemplary connector device with a net-type configuration according to an aspect of the present invention;

FIG. 2b shows a schematic view of an exemplary connector device with a net-type configuration according to an aspect of the present invention;

FIG. 2c shows a schematic view of an exemplary cell of a net-like tubular device according to an aspect of the present invention;

FIG. 2d shows a schematic view of an exemplary cell of a net-like tubular device according to an aspect of the present invention;

FIG. 2e shows a schematic view of an exemplary connector device with a square cut tubular configuration according to an aspect of the present invention;

FIG. 2f shows a schematic view of an exemplary angle of an angled proximal opening according to an aspect of the present invention;

FIG. 2g shows a schematic view of an exemplary connector device with an angled body according to an aspect of the present invention;

FIGS. 3a, 3b and 3c show schematic views of an exemplary connector device in different states according to an aspect of the present invention;

FIG. 4a shows a schematic view of an exemplary connector device with a blood vessel inserted in the device according to an aspect of the present invention;

FIG. 4b shows a schematic view of an exemplary connector device with a blood vessel everted over the device according to an aspect of the present invention;

FIG. 5 shows a schematic view of an exemplary connector device with a blood vessel everted over the device and a biological structure to be connected to the blood vessel according to the an aspect of the present invention;

FIG. 6 shows a schematic view of an exemplary biological structure attached to the external surface of a connector device and overlying a blood vessel, wherein the underlying blood vessel is retained in the device and everted over the device according to an aspect of the present invention;

FIG. 7 shows a schematic cross-sectional view of an exemplary biological structure attached to the external surface of a connector device and overlying the end of a blood vessel everted over the device according to an aspect of the present invention;

FIG. 8a shows a schematic view of an exemplary connector device with a blood vessel inserted in the device according to an aspect of the present invention;

FIG. 8b shows a schematic view of an exemplary end to end connection with a connector device according to an aspect of the present invention;

FIG. 8c shows a schematic view of an exemplary end to side connection with a connector device according to an aspect of the present invention;

FIG. 8d shows a schematic view of an exemplary end to side connection with a connector device according to an aspect of the present invention;

FIG. 9a shows a schematic view of an exemplary connector device with a biological structure inserted in the device according to an aspect of the present invention;

FIG. 9b shows a schematic view of an exemplary end to end connection with a connector device according to an aspect of the present invention;

FIG. 10a shows a schematic view of an exemplary connector device with a biological structure inserted in the device according to an aspect of the present invention;

FIG. 10b shows a schematic view of an exemplary end to side connection with a connector device according to an aspect of the present invention;

FIG. 11a shows a schematic view of an exemplary restraining ring according to an aspect of the present invention;

FIG. 11b shows a schematic view of an exemplary connector device with an exemplary restraining mechanism according to an aspect of the present invention;

FIG. 11c shows a schematic view of an exemplary end to side connection with an exemplary connector device featuring an exemplary restraining mechanism according to an aspect of the present invention;

FIG. 11d shows a schematic view of an exemplary end to side connection with an exemplary connector device restrained by an exemplary restraining mechanism according to an aspect of the present invention;

FIG. 11e shows an alternative schematic view of an exemplary end to side connection with an exemplary connector device restrained by an exemplary restraining mechanism according to an aspect of the present invention;

FIG. 12a shows a schematic view of an exemplary connector device with a biological structure inserted in the device according to an aspect of the present invention;

FIG. 12b shows a schematic cross-sectional view of an exemplary end to side connection with a connector device according to an aspect of the present invention;

FIG. 12c shows a schematic view of an exemplary end to side connection with a connector device according to an aspect of the present invention;

FIG. 12d shows a schematic cross-sectional view of an exemplary end to end connection with a connector device according to an aspect of the present invention;

FIG. 12e shows a schematic view of an exemplary end to end connection with a connector device according to an aspect of the present invention;

FIG. 13 shows a schematic view of two exemplary connector devices for connecting a plurality of structures according to an aspect of the present invention;

FIG. 14 shows a schematic view of two exemplary connector devices connecting a plurality of structures according to an aspect of the present invention;

FIG. 15 shows a schematic view of two exemplary connector devices connected to respective ends of a length of a biological or non-biological structure according to an aspect of the present invention;

FIG. 16 shows a schematic view of two exemplary connector devices joining a length of a biological or a non-biological structure to at least one other structure according to an aspect of the present invention;

FIG. 17a shows a schematic view of two exemplary net-like devices of the present invention employed in a bypass type connection according to an aspect of the present invention;

FIG. 17b shows a schematic cross-sectional side view of two exemplary net-like devices of the present invention employed in a bypass type connection according to an aspect of the present invention;

FIG. 18 shows a flow chart of an exemplary method of using an exemplary connector device according to an aspect of the present invention;

FIG. 19 shows a flow chart of an exemplary method of using a plurality of exemplary connector devices according to an aspect of the present invention;

FIG. 20 shows a flow chart of an exemplary method of production of an exemplary connector device according to an aspect of the present invention; and

FIG. 21 shows a flow chart of an exemplary method of production of a tubular net-like connector device according to an aspect of the present invention.

### DETAILED DESCRIPTION

In one aspect the present invention is of a medical connecting device. The connecting device may be a device for joining body tissues. The device may be for joining tissues in humans, mammals and animals. The device may be for joining biological tissues found internally. It is envisioned that the present invention may be used in the joining of any suitable tissues and body structures in any suitable area of the body. The device may be for joining tubular structures. The device may be for end to end connection. The device may be for end to side connection. The device may be for side to side connection. The device may also be used in the repair of a severed tissue or any suitable opening, such as a hole, a rip, a gash, a cut, a rupture or a damaged tissue. In a further aspect the present invention provides a method of using a connecting device. In an additional aspect, the present invention provides a method of using a plurality of connecting devices to connect a plurality of structures. The present invention provides uses of such a device, for anastomosis, surgery, wound treatment and a combination thereof. In a still further aspect the present invention is of a method of production of the connecting device. The present invention provides a system for replacing and/or augmenting suturing in a bypass procedure, wherein the system may include a plurality of connector devices of the present invention.

The device of the present invention is relatively facile to use and facilitates a method of joining biological tissues/s, which is fast and less time consuming than the commonly used method of suturing. The device of the present invention provides uniform contact between the structures to be joined and holds the structures together effectively. Further, the device of the present invention may provide the angle of the connection. Moreover, the device of the present invention is configured to reversibly radially expand according to the dimensions of the inserted structure and therefore does not exert substantially any external pressure on the biological structure and does not restrict or hinder the flexibility of the biological structure. The lack of pressure exerted by the device on the structures to be joined may avoid causing narrowing of the joined biological structure/s and/or other structures. The anchoring of the biological structure or other structure accommodated within the device may be provided by the dimensions of the device, which may conform to the dimensions of the inserted structure. The device is configured so that the fixing to the device of the structures to be joined does not restrict the flexibility of the structures. In addition, the means for fixing the structures to the device and facilitating contact between the structures to be connected is configured so that the join between the structures is optimally sealed, preventing leaking.

As used herein the term ‘braid’ may include, but is not limited to lengths and/or strips of for example filaments of suitable dimensions interwoven in a diagonally overlapped pattern.

As used herein the term ‘tubular braid’ may include any suitable configuration, with properties similar to a Chinese finger trap, wherein the configuration reversibly facilitates radial enlargement when the extremities are pushed inwards and reversibly facilitates radial contraction when the extremities are pulled outwards and/or pushing forces are removed. The term includes any tubular braid with suitable dimensions and suitably configured for use on a biological tissue. The term ‘tubular braid’, ‘braided sleeve’, ‘tubular plait’, and ‘plaited sleeve’ may be used interchangeably.

As used herein the term ‘tubular net’, or ‘tubular net-like’ may include any suitable configuration which facilitates reversible radial enlargement, wherein the radius may expand and return to the original size.

As used herein the term ‘radial enlargement/expansion’ may include, but is not limited to an increase of the radius and/or diameter of a tubular structure or similar shaped structure without external addition or deletion to the original structure.

As used herein the term ‘reversible radial enlargement/expansion’ may include, but is not limited to a return of the size of the radius towards and/or to its original radius on removing the forces facilitating the radial enlargement. The term may include a return to a radial size which is a reduction of the radial enlargement, but which may not be the same size as the original radius.

As used herein the term ‘radial contraction’ may include, but is not limited to a decrease of the radius and/or diameter of a tubular structure or similar shaped structure without external addition or deletion to the original structure.

As used herein the term ‘reversible radial contraction’ may include, but is not limited to a return of the size of the radius towards and/or to its original radius on removing the forces facilitating the radial contraction. The term may include a return to a radial size which is an increase of the size of the radial contraction, but which may not be the same size as the original radius.

As used herein the term ‘joining’ may include, but is not limited to contacting and connecting in any suitable way in order that a plurality of structures or parts thereof are attached. The term may include connecting the two parts of a biological structure resulting from severing or cutting of the original uncut biological structure, such that the joining will reform the original uncut biological structure. The term may include connecting two previously unconnected structures.

As used herein the term ‘biological structure’ may include, but is not limited to any suitable biological tissue, which can be accommodated in the device of the present invention. The two sides of a cut biological structure may be referred to in some examples as two separate biological structures.

As used herein the term ‘non-biological structure’ may include, but is not limited to any suitable structure which is not found naturally in a human or animal body and/or whose source is not biological. Non-limiting examples of non-biological structures may include catheters, tubes, synthetic grafts, wires, inlets of machinery, outlets of machinery and combinations thereof.

As used herein the term ‘external biological structure’ may include, but is not limited to any suitable structure, which may be biological taken from an external source from the host. Non-limiting examples of external biological structures may include biological structures or parts thereof from a different host or grown in vitro or grafts for transplanting.

As used herein the term ‘pulling’ may include, but is not limited to a tugging force applied to the device in the direction from the midline of the device towards an opening/s of the device. The term may include a pulling force applied to one section, or a plurality of sections of the device, or parts of a section of the device.

As used herein the term ‘pushing’ may include, but is not limited to a force applied to the device, where the force is applied in the direction from an opening/s of the device towards the midline of the device. The term may include a force applied to the device in the direction from an opening towards the second opening of the device. The term may include a pushing force applied to one section or a plurality of sections of the device or parts of a section of the device.

As used herein the terms ‘eversion’, ‘evert’, ‘everted’, ‘everting’ refers to the turning inside out of an end/opening of a structure, such as a biological structure or a synthetic graft, so that the inside layer is exposed. The eversion may be performed over a surface such as the opening or an edge of the opening or part thereof of the device of the present invention.

The principles and operation of a device, such as a connector according to the present invention may be better understood with reference to the figures. The figures show non-limiting aspects of the present invention.

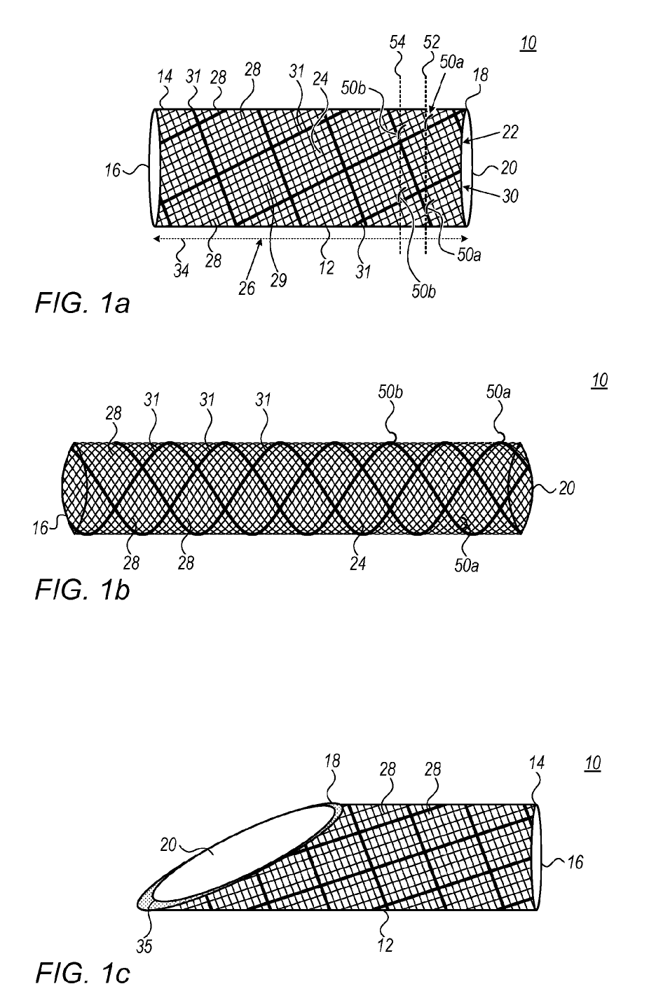

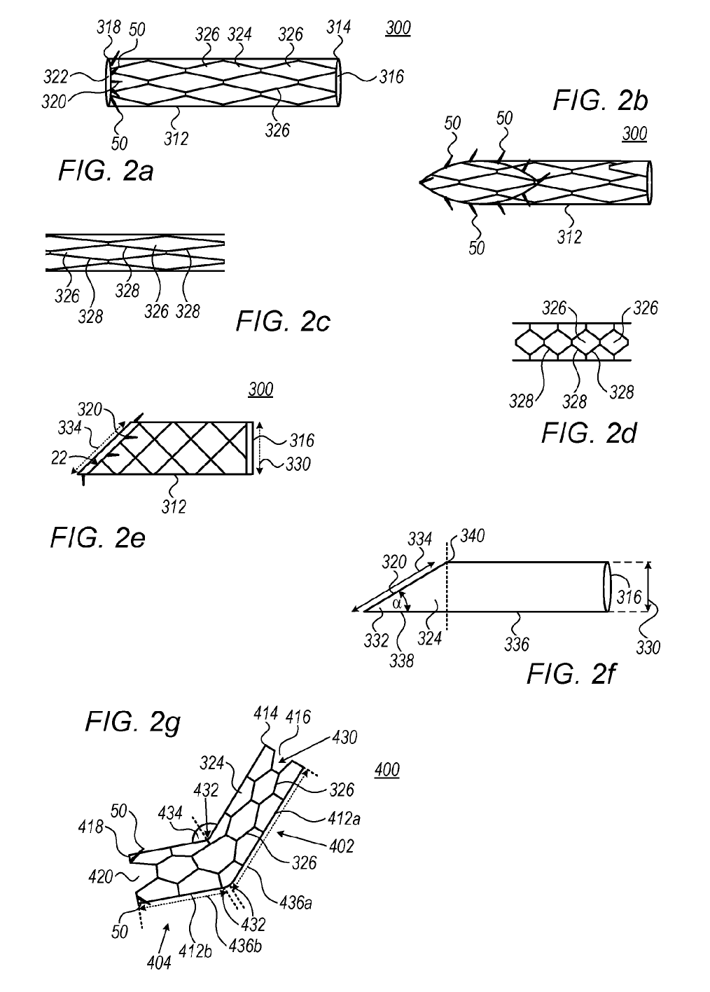

FIG. 1a, FIG. 1b, FIG. 1c, FIG. 2a, FIG. 2b and FIG. 2e show schematic views of a device according to aspects of the present invention. The device may be of a Chinese finger cuff configuration, stent-like configuration, a weave, a mesh, a network, a knit, a braid, a helically wound braid, biaxial braid, coils, spirals, knots, twists, a scaffold, a crocheted design and combination thereof or any suitable equivalent construction design. The device may be of any suitable construction design which is configured for reversible radial enlargement. The device may have a configuration, which promotes reversible radial expansion. FIGS. 1a-1c show an embodiment of a device 10 with a tubular braid configuration and FIGS. 2a, 2b and 2e show an embodiment of a device 300 with a tubular net configuration.

Referring to FIGS. 1a-1c, in one aspect the device 10 may be a tubular open ended device. The device 10 may be a right circular hollow cylinder. The device may include a longitudinal axis and a radial axis substantially perpendicular to the longitudinal axis. The device 10 may be a straight device 10. The device 10 may be flexible. The device 10 may include an elongated body 12, which may include a first distal extremity 14 with a first distal opening 16 and a second proximal extremity 18 with a second proximal opening 20. The first distal opening 16 may be oppositely disposed to the second proximal opening 20. In some embodiments, the opening 16 at the distal extremity 14 may be defined as the opening through which a structure to be joined may be inserted and the opening 20 at the proximal extremity 18 may be defined as the opening where the end of the structure to be joined is positioned and which may provide an edge over which to evert the end of the structure. The device 10 may be a hollow elongated construction including an inner surface and a lumen defining a cavity 22, which may be substantially hollow extending longitudinally between the distal opening 16 and the proximal opening 20. The central cavity 22 may be configured to receive and retain in the cavity 22 a structure such as a biological structure, a synthetic structure or any other suitable structure, which is to be joined using the device 10 of the present invention. The central cavity 22 may be configured to receive and retain only one biological structure. The dimensions of the device 10 and the internal cavity 22 therein may be constructed to optimally accommodate an inserted structure. The structure to be jointed may be a tubular structure. The end of the structure to be joined may be, but is not limited to a cut end, a severed end, a damaged end and an extremity. The device 10 may be used to join the end of the inserted structure to the end or side or any other position of any suitable opening of a biological structure, a synthetic structure or any other suitable structure.

The device 10 may be made from a flexible material, which may be constructed in a configuration and with suitable dimension such that when a structure is inserted into the cavity 22 and any pushing force applied for facile insertion is removed, the device 10 may hold tightly the inserted structure within the cavity 22. When a first end 14 and/or the second end 18 of the device 10 are pulled, the diameter of the device may narrow to hold the inserted object/s tighter within the cavity 22. The material and configuration of the material forming the device 10 facilitates reversible radial enlargement of the tubular member in response to a pushing force being applied inwards to at least one of the first end or the second end of the tubular member, the pushing force acting in the suitable direction of the longitudinal axis. The material and configuration of the material forming the device 10 facilitates reversible radial contraction in response to a pulling force, such as when the device is pulled outwards, being applied to at least one of the first end or the second end of the tubular member, the pulling force acting in the suitable direction of the longitudinal axis. Such radial enlargement and radial contraction may provide anchoring of a structure, such as not limited to a biological structure which is inserted into the cavity of the device. The device may tighten about the inserted structure when the pushing force is eliminated, or a pulling force is applied, or the inserted structure is displaced. The material of the device 10 may be configured such that the radial enlargement and radial contraction are reversible when the pushing and pulling forces are absent, such as when no structure is inserted into the cavity. The material and properties of the device 10 may be configured so that the shape of the device 10 conforms to the shape of a biological or non-biological structure retained in the device 10. The device 10 may be made from any suitable material. The material may be biocompatible and non-toxic. The material may be a material which has sufficient properties of flexibility to impart flexibility on the device. Non limiting examples of materials which may be used include nitinol, alloys of titanium and nickel, stainless steel, nirosta, platinum, gold, silver, copper, zinc, ceramic, polytetrafluoroethylene (PTFE), polyethylene, urethane, silicone, nylon, polyester, polypropylene, fabric, gut, tissue graft and combinations thereof. The device may be constructed from a tubular braid made from one material or a combination of materials. The device may be made from a material suitable for remaining long term in the body. The material may be biodegradable and/or bioabsorbable. In a device which is biodegradable, the device may be metabolized or broken down by a suitable process and the resulting broken down components may be removed from the body by for example excretion. In some embodiments, wherein long term reinforcement is needed at the point of connection, the device may be made from a material which is not bioabsorbable. The device material may be disposable. The device material may be absorbent or non-absorbent. The device material may be porous and may have different porosities. The device material may be configured to include interstitial spacing for relative movement of the components, such as the filaments or strips of the device.

The braid 26 may be similar to the tubular braid of a Chinese finger cuff. The braid 26 may be constructed from a plurality of parts or lengths 28, which are braided or interwoven. Individual parts 28, may be strips or tubes or threads or wires or filaments or any suitable form which may be braided or weaved and used to form a tubular braid or weave. As used herein the term ‘filament’ may include any suitable individual part of a braid, which can be braided, plaited or weaved. The term includes tubes, threads, wires and strips and combinations thereof. The filaments 28 may be round, flat, ribbon, square or a combination thereof. In the example shown in FIG. 1b, the filaments 28 are round filaments. Round filaments 28 may impart greater flexibility on the resulting braided device 10. The dimensions of the filaments 28 may determine the size and the flexibility of the device 10. The parts or filaments 28 may be of any suitable thickness, such that the parts or filaments 28 can be braided or formed into the reversible radial size configuration of the device. The thickness of the filament 28 to be used may be determined and optimized for each material used. The width of the filament 28 may relate to the size of the device. As such, when the size of a device is predetermined, the widths of the filaments 28 may be calculated accordingly. The radius of the device may be increased or decreased by using a greater or lesser number of filaments 28. The thickness, width and length of the filaments 28 may vary. In one embodiment, some of the filaments 31, which may have additional functions may be thicker and/or wider than the other filaments 28.

The length of the filaments 28 may be any suitable length. The length may be determined so that the device is sufficiently long to hold the inserted biological or non-biological structure. The length may be determined so that the dimensions of the device are sufficient to hold the structure to which the inserted biological or non-biological structure is to be connected to. The length may be determined so that the device will be sufficiently long to provide the needed radial expansion and contraction. The length may be calculated so that the device will be as short as possible for easy insertion of the biological or non-biological structure within the device. In addition, the length may be optimized for insertion by endoscope. The spring of the device may be determined by for example the properties of the materials used to make the device and the dimensions of the materials used. The spring may be a measure of the ability of the device to regain its original shape after being compressed or extended. The spring of the device is important, to facilitate a device with reversible sizing. The tubular braids used for protecting hoses and wiring, are not suitable for use in biological tissues, because the spring is not sufficient. The tubular braids designed for these other uses are not designed with a resilient spring, but are constructed so that they are flexible and can fold. This is achieved by the braiding of parts, wherein each part is made up of multiple filaments. Using a part of multiple filaments facilitates greater bending flexibility, but reduces the ability of the device to return to its initial state, thereby preventing the reversibility of the reversible contraction and expansion properties of the end device. The braid of the device of the present invention may be made from suitable parts, which are single filaments. In some non-limiting examples wherein the device of the present invention has a sufficiently large diameter, at least one individual part, which includes multiple filaments may be used, providing that the resulting flexibility facilitates sufficient reversible contraction and expansion.

Any suitable number of parts may be used. The number of parts may be an even number. The number of filaments 28 may facilitate the shape of the device. The number of filaments 28 may be determined according to the size of the structure to be inserted into the device. For a structure with a larger radial diameter more filaments 28 may be used than for a structure with a smaller radial diameter. The diameter of the device 10 may be greater than about 1 mm. In an aspect, wherein the device 10 of the present invention is used for accommodating a biological structure which is tubular in shape, such as for example a blood vessel, the device may be constructed in a similar shape to the biological structure. In the device 10 of tubular design, the lengthways strips, or the filaments, or the strips, or the filaments running in one direction may be referred to as ‘warps’ and the strips, or the filaments positioned over and under the warps and running in an opposing direction at an angle to the warps may be referred to as ‘wefts’. The angle between the warps and the wefts may be changed by pulling and pushing the ends of the device. When the angle 29 between the warp and weft filaments is reduced by pulling the device 10, the radius and diameter 30 of the tubular design is reduced. When the angle 29, between the warp and weft strips or filaments is increased by pushing the device 10 inwards, the radius and diameter 30 of the tubular design is increased. A cell of a braid may refer to part of the braid featuring four filaments 28 positioned over and under each other with one spacing in the center.

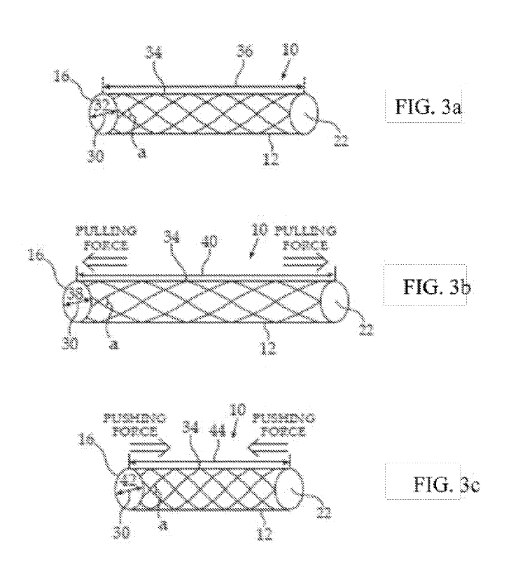

The diameter 30 of the device 10 may be changeable. The device 10 may have at least three different states, a relaxed state as shown in FIG. 3a, a pulled state as shown in FIG. 3b and a pushed state as shown in FIG. 3c. A ‘relaxed state’ refers to a state wherein the device is not pulled or pushed at its extremities and/or along its length. A ‘pulled state’ refers to the device wherein at least one of the extremities and/or a suitable part along the length of the device is pulled. A ‘pushed state’ refers to the device wherein at least one of the extremities and/or a suitable part along the length of the device is pushed. In FIG. 3a wherein the device 10 is in a relaxed state, the diameter 30 of the elongated member 12 and cavity 22 may be a first size 32 and the length 34 of the elongated member 12 may be a first size 36. The angle between warp and weft is ‘a’. In FIG. 3b wherein the device 10 is in a pulled state, the diameter of the elongated member 12 and cavity 22 may be substantially reduced resulting in a reduced diameter 38 and the length 34 of the elongated member 12 may be increased to increased length 40. The angle ‘a’ between the warp and weft is decreased compared to the angle ‘a’ in the relaxed state. FIG. 3b shows the direction of a pulling force. In a pushed state, shown in FIG. 3c, the diameter 30 of the member 12 and cavity 22 may be substantially increased to increased diameter 42 and the length 34 of the elongated member 12 may be reduced to reduced length 44. The angle ‘a’ between the warp and the weft is increased compared to the angle ‘a’, in the relaxed state. The type of configuration used to construct the device 10 may include any suitable construction, which will facilitate such a substantially reversible change in size as shown in FIGS. 3a-3c. In the non-limiting example shown in FIG. 1a and FIG. 1b the device 10 is constructed as a tubular braid for facilitating the reversible change in size.

The device 10 may be configured from a shape-memory alloy, such as but not limited to nitinol. The device may be designed with an original relatively rigid configuration such that the radius is of suitable dimensions for optimal fit of a biological structure/s to be treated. Before use, the device may be cooled to a temperature below the critical temperature of nitinol. At such a temperature, the alloy is flexible and can be bent easily into any suitable shape for easy insertion of the biological and/or non-biological structures into the device. When the device warms up to body temperature, the nitinol is heated to its critical temperature and transforms into its original rigid arrangement. As such, using a change in temperature, the device may be configured for reversible radial enlargement and reversible radial contraction. The device 10 may use a combination of the shape memory properties of for example nitinol and the reversible expansion and contraction properties of the braid or the reversible expansion properties of the net configuration.

The device 10 may be used for application to blood vessels, lymphatic vessels, ligaments, tendons or nerves or any other suitable biological structure of any suitable size. The device 10 may be effective for application to tubular shaped biological and non-biological structures. The connector devices 10 of the present invention may be designed and constructed in a range of dimensions, to be suitable for different biological structures and different ranges of size of biological structures. In some embodiments, the connector device 10 is sufficiently expandable for use with different sizes of biological structure.

The device 10 may have a permanent set put into it so that it is normally open with the larger diameter associated with the ‘pushed state’ described above. In such a non-limiting example when it is pulled, the device will collapse down to the diameter of the biological structure inserted within the device. Optionally, the device 10 may have an over tube which is slideable and which facilitates the ‘pulled state’ and ‘pushed state’.

Referring to FIG. 2a and FIG. 2b, which show schematically embodiments of an exemplary device 300 of the present invention featuring a tubular mesh or net type configuration. In an embodiment of a net-type tubular configuration, the device 300 may be configured for reversible radial enlargement. Internally applied radial forces, such as resulting from a structure inserted into the cavity of a net-like device may result in reversible radial enlargement of the device. In contrast to the braid configuration, the tubular net configuration may not be configured for substantial reversible radial contraction to result in a reduction of the original size of the radius. The net structure may be constructed to provide a sufficient degree of flexibility to the tubular device 300 to promote reversible radial expansion. The radial enlargement may result in reversible changes in the linear dimensions of the device 300. However, unlike the braided configuration, the design of the net facilitates relative linear rigidity and does not facilitate pulling and pushing of the extremities, or pulling and pushing of the extremities to provide substantial reversible linear expansion and linear contraction.

The device 300 may be a tubular open ended device 300. The device 300 may include a longitudinal axis and a radial axis substantially perpendicular to the longitudinal axis. The device 300 may be a straight device 300. The device may be a right circular cylinder, wherein the cylinder is hollow. The device 300 may include an elongated body 312, which may include a first distal extremity 314 with a first distal opening 316 and a second proximal extremity 318 with a second proximal opening 320. In some embodiments, the distal opening 316 and the proximal opening 320 are parallel to each other. In some embodiments, the distal opening 316 and the proximal opening 320 are congruent. In some embodiments, the opening 316 at the distal extremity 314 may be defined as the opening through which a structure to be joined may be inserted and the opening 320 at the proximal extremity 318 may be defined as the opening where the end of the structure to be joined is positioned and which may provide an edge over which to evert the exposed end of the structure. The device 300 may be a hollow elongated construction with a cavity 322. The cavity 322 may be for receiving and retaining a biological or a non-biological structure. The cavity 322 may be for receiving and retaining only one biological or non-biological structure. The body 324 of the device 300 is constructed as a net 324 featuring an array of connected net cells 326. The tubular device may 10 include any suitable shape and design of net structure. The cells 326 may have any suitable shape and dimensions. The shape and dimensions of the interconnected cells 326 may affect the flexibility and radial expansion properties of the device 300. The cells of the net may be constructed in a shape to give maximal radial expansion. A shape of a cell 326 with longer sides 328, such as long sided open diamond shaped cells as shown in FIG. 2c can result in greater reversible expansion to provide a wider tubular member than a cell 326 with shorter sides 328 as shown schematically in FIG. 2d. Suitable shapes of cells may include a net featuring an array of connected rings, diamonds, squares, rectangles, triangles, polygons and combinations thereof. In some embodiments, each cell 326 of the array is connected to each of the surrounding or bordering cells 326. In one embodiment, some of the cells 326 in the array may not be connected to all surrounding or bordering cells 326.

The tubular net configuration may be formed by a laser cut or any other suitable cut of a tubular member. Laser cutting may provide a combination of cell designs, spacing and sizing in the same device. Such a combination may be difficult to achieve with a braid configuration, which cannot be constructed by laser cutting. FIG. 2a shows the non-limiting example of an array of connected diamond shaped cells 326. The net structure may be constructed from cells 326 of the same size or may include a plurality of sizes of connected cells 326. In some embodiments, the part of the tubular member 300 adjacent to the proximal opening 320, may include smaller sized cells 326. Smaller cells 326 may provide more surface onto which protrusions 50 can be applied, or from which protrusions can be formed, such as spikes.

FIG. 2e shows a schematic view of an exemplary alternative configuration of a net-like device 300 according to an aspect of the present invention. The device 300 shown in FIG. 2e may feature a square cut tubular net-like device, wherein the flexible tubular member 312 features a rectangular shaped distal opening 316 and a rectangular shaped proximal opening 320 and a rectangular shaped cavity 322. A square cut device 300 with a proximal opening 320 cut at an acute angle to the longitudinal axis of the device may provide a greater proximal opening 320 to diameter 330 ratio. Insertion of a tubular biological or non-biological structure into the cavity 322, may reversibly expand the cavity 322 into a more circular tubular form, such as is the natural form of the regular tubular net-like device described in FIG. 2a and FIG. 2b.

The net-like device 300 may be made from any suitable material, such as described herein for the braided configuration 10. In some embodiments, the net-like device 300 may be made from nitinol. The net-like configuration may facilitate a more open structure than a braided configuration. The length of the net-like device may be determined so that the device is sufficiently long to hold part of the biological or non-biological structure inserted and accommodated in the cavity. The length may be determined so that the dimensions of the device are sufficient to hold the structure to which the inserted biological or non-biological structure is to be connected to. The length may be determined so that the device will be sufficiently long to provide the needed radial expansion. The length may be calculated so that the device will be as short as possible for easy insertion of the biological or non-biological structure within the device. In addition, the length may be optimized for insertion by endoscope. The spring of the net-like device may be determined by for example the properties of the materials used to make the device and the dimensions of the materials used. The spring may be a measure of the ability of the device to regain its original shape after being extended. The spring of the device is important, to facilitate a device with reversible sizing.

Referring to FIG. 1a and FIG. 2a, the respective devices 10, 300 include a proximal opening 20 and a distal opening 16 (proximal opening 320, distal opening 316 in device 300). In order to avoid repetition, the description will refer to the numbering and components in FIG. 1a, but apply to the similar components of device 300 show in FIG. 2a. The proximal opening 20 and the distal opening 16 may be perpendicular to the length 34 of the tubular member as shown in FIG. 1a. The entire proximal opening 20 may be perpendicular to the length 34 (longitudinal axis) of the tubular member 10. The resulting perpendicular opening 20 provides a suitable means for facilitating an edge over which to evert an end of a biological structure or other structure so the opening of the exposed end of the inserted structure is substantially perpendicular to the length 34 of the tubular member 10. The perpendicular proximal opening 20 of the tubular member 10 may be configured so that a structure to be joined to a structure retained in the cavity of the device, whose end is everted over the proximal opening 20 is positioned substantially about one hundred and eighty degrees end to end to the structure retained in the cavity of the device, for end to end joining of the structures. The proximal opening 20 may be parallel to the distal opening 16. The proximal opening 20 and the distal opening 16 may be similarly sized and shaped.

In some embodiments, the proximal opening 20 and/or the distal opening 16 or part thereof of the device 10 may not be perpendicular to the length 34 (longitudinal axis) of the tubular member device 10 and/or may not be uniform as shown schematically in FIG. 1c, FIG. 2b and FIG. 2e. In some embodiments, the proximal opening 20 may be different from the distal opening 16. In some embodiments the proximal opening 20 may have different configurations according to the use of the device 10. The proximal opening 20 may provide the shape of the edge to an end or opening of a biological structure or non-biological structure to be joined. The shape of the proximal opening 20 may provide the angle of attachment of a biological structure or non-biological structure to be joined. An angled proximal opening 20 may be configured for joining structures, wherein the openings/ends of the structures are not positioned at substantially one hundred and eighty degrees from each other. A device 10 with an angled or non-uniform shaped proximal opening 20 may be suitable for use in a procedure to provide certain predetermined angled joins, which are repeatable without the need for precise cuts of the biological structures and non-biological structures to be joined. In some examples and procedures the cut of the biological structure and/or non-biological structure may not be a straight perpendicular cut and/or it may not be desirable for an everted edge of the biological structure and/or non-biological structure or part thereof to be perpendicular to the longitudinal axis 34 of the tubular member 10. The proximal opening 20 may be constructed to provide the correct angle and/or shape for different types of joining and for connecting different shapes of openings and ends of biological structures and other structures. In one non-limiting example a tubular member device 10 with an angled or non-uniform shaped proximal opening 20 may be suitable for use in a bypass procedure. The proximal opening 20 may be any suitable shape, such as but not limited to rounded, elliptical, oval, straight edged, square, triangular, kite shaped, poly-sided and a combination thereof. In some embodiments, the proximal opening 20 may be oval shaped. The proximal opening 20 may include a plurality of sides of a plurality of different lengths, which are at angles to each other. The shape of the proximal opening 20 may be carved out from the body 12 of the tubular member device 10 to result in part of the tubular body 12 being an incomplete ring.

FIG. 2b shows an exemplary embodiment of a net-like tubular device 300, wherein the proximal opening 320 is different from the distal opening 316. The distal opening 316 is made by a ninety degrees cut relative to the longitudinal axis of the tubular member and the distal opening is perpendicular to the length of the device 300. The proximal opening 320 and the distal opening 316 may be of any suitable size. The criteria for the size of the distal opening and the size of the proximal opening may be different. A distal opening 316 may be sized for insertion of a tubular structure through the distal opening 316 and for the resulting tubular radius to sufficiently hold and retain a structure in its cavity 322. The non-stretched radius of the device 300 or the stretched radius may fit and hold the radius of a tubular biological structure. In an example of a device, which may be used with a variety of different sized structures, it is desirable that the original size of the radius of the device is small enough to hold a relatively thin tubular structure. It is advantageous that the proximal opening 320 of the device 300 is relatively large for increased surface area of the area of connection, increased blood flow at the connection and easier manipulation by a user for more facile and optimal joining. If the proximal opening 320 is at ninety degrees to the longitudinal axis, it will be restricted in size by the diameter of the device 300 and to the same dimensions as the distal opening 316 and the tubular part of the device 300. Cutting the proximal opening 320 to form an angled proximal opening 320 relative to the longitudinal axis of the device 300 facilitates a much larger opening 320 and provides all the advantages of such an opening 320 without affecting the radial dimensions of the tubular part of the device 300, which are sized to optimally hold a tubular biological or non-biological structure.

It may be desirable to have a larger proximal opening 320 for providing a larger connection surface area of the structures to be joined with the device 300. A cut at a more acute angle 332 (shown schematically in FIG. 2f), which cuts into part of the body 324 of the device 300 provides an angled proximal opening 320, which is substantially larger than the diameter 330 of the device 300. In some non-limiting examples the length 334 of the angled opening 320 may be from about twice to about ten times the diameter 330 of the device 300. In one non-limiting example the ratio of the diameter 330 of the device 300 to the maximal length 334 of the angled proximal opening 320 is at least one to five. In a square cut device such as shown in FIG. 2e, the ratio of the maximal length 334 of an angled proximal opening 320 to the diameter of the device 330 is larger than in a regular circular cut tubular device 330, such as shown in FIG. 2b. Use of this property of the rectangular cut device may be effective when joining biological or non-biological structures with a small radius.

In a net-type tubular device 300, the angled opening 320 may be achieved by cutting with a laser. In some embodiments, the proximal opening 320 is formed by cutting the body 324 of the device 300 at an acute angle 332 of from about ten degrees to about forty degrees. In one example, the proximal opening 320 is formed by cutting the tubular member 312 at an angle 332 of about fifteen degrees. The large angled opening 320 confers a degree of linear rigidity on the device. As shown schematically in FIG. 2f a device in which the proximal opening 320 is carved out of part of the tubular body 324, results in a device 300 wherein a first part of the tubular device may have a complete ring structure 336 and a second part 338 of the tubular device has an incomplete ring structure 338. The complete ring structure 336 may feature a right circular hollow cylinder. In a device with an angled opening, the device may include a complete tubular part 336 of the device of connected open cells in a net-like design, which extends longitudinally from the distal opening 316 until the nearest point 340 of the proximal opening 320 in relation to the distal opening 316. The tubular body second part 338 of the device may be continuous with the complete tubular part 336. The tubular body second part 338 may be in line with the tubular body first part 336 and may feature the same longitudinal axis as the tubular body first part 336. The tubular body second part 338 may feature an incomplete hollow cylinder, the proximal opening carved out from the body of the cylinder at an acute angle to the longitudinal axis to form an incomplete ring of connected open cells in a net-like design in a radial line with the proximal opening 320. The complete tubular part 336 of the device 300 may be sized for optimal use by a user, which may include easy holding of the device 300. In some non-limiting examples, the length of the complete tubular part 336 of the device may be from about 10 mm to about 25 mm. In one example, the length of the complete tubular part 336 of the device is about 15 mm. In a tubular net-like configuration, the device may reversibly radially expand according to the dimensions of a structure inserted and accommodated within the cavity of the device 300. The size of the angled proximal opening 320 of a tubular net-like device may stay substantially the same and not expand when the tubular body reversibly expands, or is in a relaxed resting state. In an example of a braided tubular configuration, cutting at an angle and carving out part of the body of the device, results in a braid with different lengths, which may affect the reversible contraction and expansion properties of the braid. An angled opening, such as a proximal opening 20 cut at an acute angle of a device 10 with a braided configuration may be constructed using a flange 35 about the proximal opening 20, such as shown in FIG. 1c. The flange 35 may be flush with the braid or may protrude from the body of the device 10. A flange 35 may be included in an example of a device 300, which is a tubular net-like device. The other components and features of the device shown in FIG. 2b are similar to the device described in FIG. 2a.

The present invention also provides a net-like device, which may be a tubular device with a continuous hollow lumen for receiving and retaining a biological structure, the device featuring an angled body 400. FIG. 2g shows an exemplary embodiment of a net-like tubular device of interconnected cells configured for reversible radial enlargement of the tubular member featuring a body, which includes a bend 400. The device 400 may include a proximal opening 420 at a proximal extremity 418 of the device 400 and a distal opening 416 at a distal extremity 414 of the tubular body. The distal opening 416 and the proximal opening 420 of the device with the bend 400 are not parallel, or in the same longitudinal line. The angled tubular body may include a longitudinal axis, which is not straight, but which has a bent configuration resulting in two different longitudinal axes 412a, 412b. The proximal opening 420 and the distal opening 416 may be perpendicular to the respective longitudinal axis 412b, 412a that the opening 416, 420 is adjacent to. The first longitudinal axis 412a may be of a first section 402 of the tubular member and the second longitudinal axis 412b may be of a second section 404 of the tubular member. The first section 402 may include the first longitudinal axis 412a substantially perpendicular to a first radial axis and in a first longitudinal line extending from the distal opening 416 to the point of bending 432, the bend inclining the longitudinal line so that it not in the same line as the first longitudinal axis and is angled away. The second section 404 may include the second longitudinal axis 412b perpendicular to a second radial axis and in a second longitudinal line with respect to the first section 402 of the device 400, the second section 404 extending from the bend at the point of bending 432 to the proximal opening 420 of the device 400. The two longitudinal axes 412a, 412b and the first section 402 and the second section 404 of the tubular member are joined at the point of bending 432 at an angle 434 to each other. The angle 434 may be any suitable angle, which may be, but is not limited to an angle 434 of less than about one hundred and eighty degrees. The device 400 features the first section of the tubular member 402 and the second section of the tubular member 404, which are integrally connected to form a tubular device with a continuous, uninterrupted lumen 430 extending from the distal opening 416 until the proximal opening 420. The dimensions of each section 402, 404 of the device 400 may be the same. In some embodiments, the dimensions of each part 402, 404, of the device 400 may be different. In some embodiments, the length 436a of the first tubular member part 400 and the length 436b of the second tubular member part 404 may be different.

A device with an angled body 400 may facilitate connection of two structures, which may be biological structures, at an angle 434 defined by the angled body of the device 400. A structure to be joined, such as a first biological structure may be inserted through the distal opening 416 of the device 400, part of the first biological structure may be held and retained in the cavity 430 of the device 400 and an extremity of the inserted first structure may be everted over the proximal opening 420. The proximal end 418 of the device 400 may be configured to be inserted into a second structure, such as a second biological structure, such that a portion of the second biological structure encircles and overlies the everted portion of the first biological structure. In some embodiments, the second section 404 of the device 400 is configured for insertion into a tubular biological structure, such as an artery, and is positioned so that the second part 404 of the device 400 is held in the lumen of the tubular biological second structure, such that the biological second structure is substantially concentric to the second part 404 of the device 400 held within the biological second structure. The first part 402 of the device 400 may protrude out of the opening in the second biological structure, such as the artery, at an angle equivalent to the angle 434 of the bend of the device 400. The everted end of the first biological structure and the second biological structure may be fixed to the device 400 by a plurality of protrusions 50 protruding out from the exterior surface of the tubular body of the device 400 as herein described. The plurality of spaced apart protrusions 50 may be for fixing to the device 400 an end of the first biological structure everted over the proximal extremity of the device 400 and for fixing the second biological structure to the external surface of the device 400 to facilitate contact between the second biological structure and the everted end of the first biological structure for connecting the first biological structure to the second structure. The protrusions 50 may be positioned on the exterior surface of the device 400 about the proximal opening 420 of the device 400. In some embodiments the protrusions 400 may be fixed at a suitable distance from the proximal opening 420 of the device 400 to minimize contact with flowing blood. The device 400 may be constructed from any suitable material as herein described. The device 400 may be constructed in any suitable configuration, which facilitates reversible radial expansion. A device with an angled body 400 may be used in an end to side connection and an end to end connection.

As shown in FIGS. 1a-1c and 2a-2g, the device 10 may include an edge about the proximal opening 20 (320 in FIGS. 2a-2g) over which a biological or non-biological structure inserted and held in the cavity of the device may be everted. The proximal opening 20 may provide the edge. In a device with a flange 35, such as shown in FIG. 1c, the flange 35 may provide the edge. Eversion may provide an exposed internal surface part of the end of the structure inserted in the cavity of the device, the end which may be fixed to the external surface 24 of the tubular member 10 with a means 50 to hold and fix an inserted biological or non-biological structure or synthetic graft to the tubular member device 10. In some embodiments, the means 50 may include a spaced apart plurality of protrusions 50, which protrude out from the external surface 24 of the tubular member device 10. The plurality of protrusions 50 may be configured to pierce the tissue of an everted part of the inserted structure and transfix it to the exterior surface 24 of the tubular member device 10 (300). The plurality of protrusions 50 may be configured for fixing to the device 10 (300) a second structure such as a biological or non-biological structure to be connected to the inserted biological or non-biological structure accommodated in the device 10 (300). The plurality of protrusions 50 may be configured for facilitating contact between the end of the inserted biological or non-biological structure and the other structure to be connected for connection of the structures. Non-limiting examples of suitable protrusions 50 include spikes, prongs, hooks and pointed extensions. The protrusions 50 may be applied to the external surface 24 of the tubular member device 10 (300). The plurality of protrusions may position the structures to be joined to facilitate contact and connection between the everted end of a structure held in the cavity of the device 10 (300) and the encircling and overlying end of the other biological or non-biological structure. In this way, the device may facilitate joining the structure held and retained in the cavity of the device 10 (300) to the other structure.

In some embodiments, the protrusions 50 may be constructed from a surface of the tubular member device. Referring to the exemplary braided device 10 shown in FIG. 1a, the protrusions may be constructed from at least one filament 28. A plurality of filaments 28 may be braided to form a tubular device 10. Some of the plurality of filaments 28 may be manipulated to form protrusions 50. The interwoven lengths configured to provide the at least one protrusion may be thicker than the plurality of interwoven lengths which are not configured to provide the at least one protrusion. In one non-limiting example, at a position for a protrusion 50 a filament 28 may be cut at that position and a hook formed. The hook or other suitable protrusion 50 may be manipulated in order that it protrudes out from the external surface 24 of the device 10 and is configured to hold a structure, such as an everted structure, or any other structure. In an alternative embodiment, protrusions may be applied to the braided device, such as by fusing, or gluing, or tying, or sewing, or using any other suitable technique.

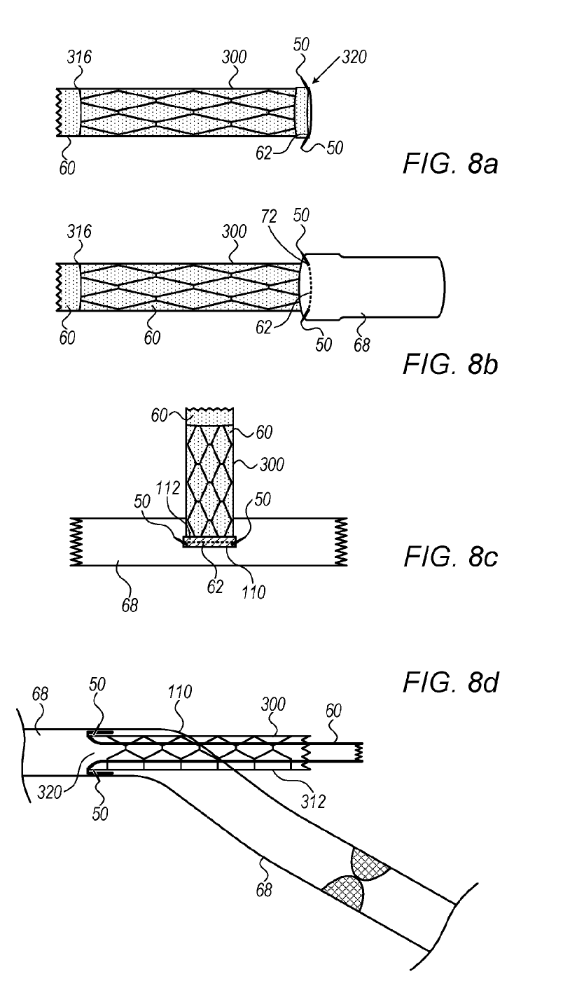

Referring to the exemplary tubular net devices 300 shown in FIG. 2a and FIG. 2b, the protrusions 50 may be formed by laser cut of the body 12 of the device 300 in a similar way to the formation of the net configuration. In such a way, the protrusions 50 may be integrally formed with the device 300. Protrusions 50, which are integrally formed may be stronger than protrusions 50, which are attached to the device. In some embodiments, a plurality of protrusions 50 are formed about the proximal opening 320 of the net-like device 300. The plurality of protrusions 50 may fix the everted end of a structure held in the cavity 322 of the device such that the exposed opening of the everted structure is configured to the same shape, angle and dimensions of the proximal opening 320 of the device 300. In some embodiments one line of spaced apart protrusions 50 is formed about the proximal opening 320. In some embodiments the same protrusions 50 are used to fix both the everted end of a structure inserted in the device and to fix the end of a structure to be connected to the structure inserted in the device. The protrusions 50 may be configured to be strong enough and sized to sufficiently pierce and hold both the everted part of the biological structure accommodated in the cavity 322 of the device 300 and the other structure. The same plurality of protrusions 50 may be employed for fixing both structures to be joined in order to avoid deep insertion of the device 300 into the opening of a biological structure. In an embodiment, wherein the device is for use in a bypass type connection, it may be desirable for the depth of insertion of the device into the opening of the artery to be minimal in order to avoid additional damage or blockage to the artery. In such an embodiment, the device may include the plurality of protrusions 50 positioned about the proximal opening of the device in order that only the proximal opening and substantially not the body of the device needs to be manipulated about the opening of the artery. In an embodiment, wherein a device with an angled body is used, such as shown schematically in FIG. 2g, or a method is used as shown schematically in FIG. 8d, part of the body of the device may be fully inserted into the opening such that the inserted part is fully encircled by the artery and is in the same longitudinal line as the artery, facilitating blood flow in that part of the artery and avoiding the problem of the device blocking blood flow in the part of the artery in which it is inserted. In such an embodiment, placement of the plurality of protrusions 50 may be determined in order to minimize contact between flowing blood and the protrusions 50.

The construction of the protrusions 50 used in the present invention may be undesirable and detrimental if used internally in for example a stent as the protrusions 50 of the present invention are sized to be long enough for optimal penetration of the tissue of the two structures to be joined and to hold both tissues.

The protrusions 50 may be configured to point in a direction away from the proximal opening 20 (220) towards the distal extremity 14 (214) of the tubular member device 10 (300). The protrusion 50 may be constructed in a form, such as, but not limited to a hook or spike, which may pierce and hold the structure onto the device 10 (300). The size of the protrusions 50 may be any suitable dimensions that facilitate transfixing the structure inserted in the device 10 (300) onto the device 10 (300). The size of the protrusions 50 may also take into account the internal environment of the body and be sized so as not to contact any other biological structures in the body in the vicinity of the site of the anastomosis or other type of joining.

The protrusions 50 may be spaced apart about the circumference of the device 10 (300). The protrusions may be equidistant from each other. The plurality of spaced apart protrusions 50 may be positioned adjacent to the proximal extremity 18 of the tubular member 10 (300) and may be positioned and angled to fix the inserted structure to the device and to fix to the device the structure to be joined to the inserted structure.

In some embodiments, such as shown schematically in FIG. 1a and FIG. 1b the plurality of spaced apart protrusions 50 may include a first plurality of protrusions 50a and a second plurality of protrusions 50b, wherein the first plurality of protrusions 50a are configured for fixing to the external surface 24 of the device 10 a biological or a non-biological structure, which is inserted into the device 10 with an end everted over a proximal edge 18 of the device 10. The second plurality of protrusions 50b may be configured for fixing to an external surface 24 of the device 10 a structure to be joined to the structure inserted and accommodated in the device 10. The first plurality of protrusions 50a and the second plurality of protrusions 50b may be positioned and configured for contacting at least part of the other structure to be joined with at least the everted part of the inserted biological or non-biological structure. The positioning of the first plurality of protrusions 50a and the second plurality of protrusions 50b may be determined in order to facilitate optimal sealing. A protrusion 50 in the first plurality of protrusions 50a and a protrusion in the second plurality of protrusions 50b may not be in the same longitudinal line. In some non-limiting embodiments, a protrusion in the second plurality of protrusions 50b may be positioned longitudinally between the longitudinal positons of two protrusions of the first plurality of protrusions 50a. The first plurality of protrusions 50a may be configured about the circumference of the tubular member 10 in a first at least one line 52 adjacent to the proximal end 18 of the device 10 and the second plurality of protrusions 50b may be configured about the circumference of the tubular member 10 in a second at least one line 54 adjacent to the proximal end 18 of the device 10 and wherein the first at least one line 52 may be nearer to the proximal end 18 of the tubular member 10 than the second at least one line 54. The first plurality of protrusions 50a may be positioned near to the proximal opening 20 of the device 10 for optimal fixing to the external surface 24 of the device 10 of the inserted structure held and retained in the cavity of the device, and facilitating an exposed open end of the inserted structure positioned at the proximal opening 20 of the tubular member 10. The nearer the first plurality of protrusions 50a to the proximal opening 20, the easier it may be for a user to evert the inserted structure and fix it with the protrusions 50a. In one non-limiting example, the first line of protrusions was disposed at about 2-3 mm from the proximal extremity of the device 10. The distance between the first at least one line 52 and the second at least one line 54 may be determined in order to facilitate flexibility of the device and the structure inserted therein. In one non-limiting example, the distance between the first at least one line 52 and the second at least one line 54 was less than about 6 mm. The two separated lines 52, 54 of the plurality of protrusions and the fixing to the device of the two structures at different spaced apart lines along the device may facilitate optimal sealing of the joined structures. The two lines 52, 54 of protrusions 50 provide an additional barrier to prevent openings in the join of the inserted structure and other structure and to prevent leaking. The device may include any suitable number of protrusions 50. In one embodiment of a device with a first plurality of spaced apart protrusions 50a and a second plurality of spaced apart protrusions 50b, such as shown schematically in FIG. 1a and FIG. 1b the number of protrusions in the first plurality of spaced apart protrusions 50a may be at least three and the number of protrusions in the second plurality of spaced apart protrusions 50b may be at least three. The number of protrusions may be determined for optimal sealing and joining of the structures fixed to the device 10.

The length 34 of the devices of the present invention may be adjusted according to the end use of the device 10, 300 and according to the type and length of the biological vessel/s to be connected. The length of the device may be determined as described hereinabove for determining the length of a filament. The length of the device may be determined so that it is long enough to hold a biological or non-biological structure accommodated therein. The length may be calculated so that the device includes a sufficient number of protrusions for fixing to the device a biological or non-biological structure inserted and retained in the device and for fixing a second biological or non-biological structure to the external surface of the device to connect the two structures. The length may be determined so that the device will be sufficiently long to provide the needed radial expansion. The length may be calculated so that the device will be as short as possible for easy insertion of the biological or non-biological structure within the device. In addition, in one non-limiting example the length may be optimized for insertion of the device into the body by endoscope.

Referring to FIG. 1a, which shows schematically an exemplary device with a tubular braid configuration, the ends of the device 14 and 18 may be uneven or may be constructed so that the individual filaments, or strips do not stick out at the end. Non-limiting examples of suitable finishes to the ends 14 and 18 are folding of the filaments into the tubular braid, tucking the ends of the filaments into the braid, gluing down the ends, stitching the ends or a combination thereof. FIG. 1b shows schematically a device 10 with uneven ends 14, 18 resulting from the ends of the braided filaments.

The device 10, 300 may include an inner surface and an outer surface. The inner surface and the outer surface may have the same structure or may have different structures. The inner surface and/or the outer surface may be coated with at least one layer of coating. The at least one layer of coating may include one substance or a combination of substances. The at least one layer of coating may include a glue, an adhesive, sealant or any suitable adhering substance, an antibiotic, an antibacterial substance, at least one substance for promoting healing, a therapeutic agent, an anticlotting substance, a clotting substance, a vitamin, an antioxidant, an anti-inflammatory agent, an anesthetic agents, an anti-coagulant, an anti-restenosis agent, a thrombosis agent, an immunosuppressant agents, a dye, a movement retardation composition or a combination thereof. The at least one layer of coating may be covered with at least one release liner. The at least one release liner may be removed prior to use. The inner surface and/or the outer surface may be coated with a different at least one layer of coating.

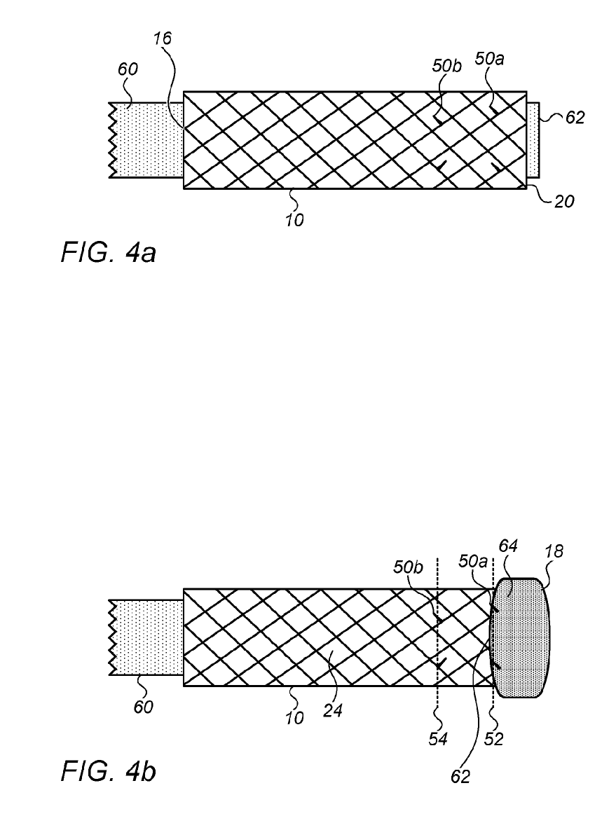

FIG. 4a, shows schematically an exemplary structure such as, but not limited to a severed blood vessel 60, inserted and held and retained in a device 10 of the present invention. The device 10 may be any device of the present invention as described herein. The severed end 62 of the blood vessel 60 may be inserted through the distal opening 16 of the device 10 into the cavity 22 of the device 10 until the severed end protrudes from the proximal opening 20 of the device with part of the blood vessel 60 adjacent to the severed end 62 of the blood vessel accommodated within the cavity of the device 10. In an example wherein the device is a braided device, the braided device may hold the blood vessel using a combination of the properties of the braided tubular member and the material the tubular member is constructed from. In a non-limiting embodiment, the tubular member is constructed from nitinol.

The severed end 62 of the inserted blood vessel 60 may be everted over the proximal edge 18 of the device 10 as shown schematically in FIG. 4b. The severed end 62 may be everted and fixed with a first line of protrusions 52. The protrusions 50 may be protrusions 50 as described herein and may pierce the blood vessel 60 adjacent to the severed end 62 and fix the blood vessel 60 to the external surface 24 of the device 10 exposing the internal surface 64 of the blood vessel 60 adjacent to the severed end 62.

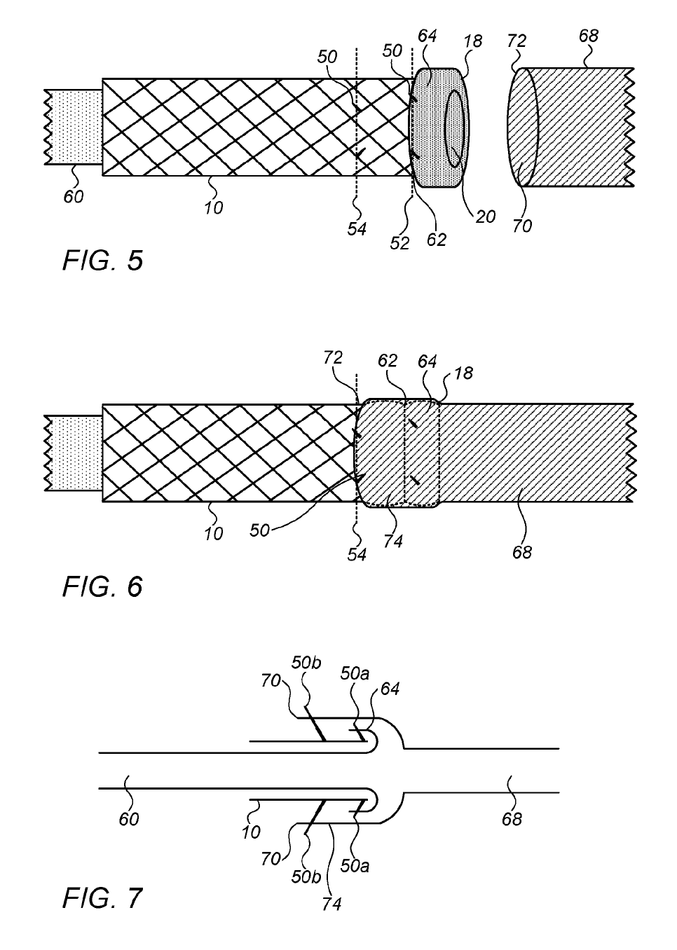

FIG. 5 shows schematically a structure 60, which may be a biological or a non-biological structure inserted and held and retained in the cavity of a device 10 of the present invention and with the severed end 62 of the inserted structure everted and fixed to a first line 52 of a plurality of protrusions 50 adjacent to the proximal opening 20 of the device 10. For illustrative purposes, which are by no means intended as limiting, the structure 60 may be referred to as a blood vessel as described for FIG. 4a and FIG. 4b. A second structure 68 to be joined to the blood vessel 60 retained and held in the cavity of the device 10 is brought into contact with the extremity of the inserted blood vessel 60 attached to the device 10. In one non-limiting example, the second structure is an artery.

FIG. 6 shows a schematic view of an exemplary second structure 68 attached to the external surface of a connector device 10 and overlying a blood vessel 60. The underlying blood vessel 60 is retained in the device and everted over the device as described in FIG. 5. As can be seen in FIG. 6, the proximal end 18 (shown as a dotted line) of the device 10 with the blood vessel 60 held and retained in the cavity of the device 10 and the severed end 62 of the blood vessel everted over the proximal opening 20 of the device, is encircled, and surrounded by the overlying first end 72 and part of the second structure 68. In one embodiment, the proximal end 18 (shown as a dotted line) of the device 10 with the blood vessel 60 held and retained in the cavity of the device 10 and the severed end 62 of the blood vessel everted over the proximal opening 20 of the device may be inserted into the opening 70 of the second structure 68. The device 10 may be inserted until the extremity 72 of the opening 70 of the second structure 68 is adjacent to a plurality of protrusions 50, which in some non-limiting examples may be a second line 54 of a plurality of protrusions. The second line 54 of protrusions may attach to the second structure 68, such as, but not limited to by piercing through the internal layer of the lumen of the second structure and fix a part 74 of the second structure 68 to an external surface of the device 10. In an alternative embodiment, the first end 72 of the second structure 68 may be pulled over the proximal end 18 (shown as a dotted line) of the device 10 with the blood vessel 60 held and retained in the cavity of the device 10 and the severed end 62 of the blood vessel everted over the proximal opening 20 of the device. The first end 72 of the second structure 68 may be pulled over the device 10 and over the everted portion 64 of the blood vessel 60 until the extremity 72 of the opening 70 of the second structure 68 is adjacent to a plurality of protrusions 50, which in some non-limiting examples may be a second line 54 of a plurality of protrusions. The second line 54 of protrusions may attach to the second structure 68 and fix a part 74 of the second structure 68 to an external surface of the device 10.

FIG. 7 shows a schematic cross-sectional view of an exemplary second structure 68 attached to the external surface of a connector device 10 and overlying the end of a blood vessel structure 60 everted over the device 10 according to an aspect of the present invention. As can be seen schematically in FIG. 7, part 74 of the second structure 68, which is fixed to the device 10 is in contact with and overlays the everted and fixed part 64 of the blood vessel 60 inserted and retained and held in the device 10, facilitating connection of the two structures 60, 68. The device 10 provides connection of a relatively large surface area of the ends of the two structures 60, 68, which in the non-limiting example shown in FIG. 6 and FIG. 7 is an end to end connection. In some examples, the device may be similarly used for an end to side connection.

In some embodiments, the device may include only one line of protrusions 50, which may be disposed on the proximal opening, such as the device shown in FIG. 2a. FIG. 8a shows schematically an embodiment of the present invention wherein a biological structure 60 is inserted into and retained and held in the cavity of an exemplary device 300 with one line of protrusions 50. The exposed end 62 of the biological structure 60 is everted over the proximal edge 320 of the device and fixed with the single line of the plurality of spaced apart protrusions 50 positioned about the external surface of the proximal opening 320. The biological structure 60 is fixed to the external surface of the device 300 exposing the internal surface of the biological structure 60 adjacent to the severed end 62. The device may have a tubular braided configuration or a tubular net-like configuration. FIG. 8a shows a device 300 with a tubular net-like configuration and with a proximal opening 320 cut at ninety degrees to the longitudinal axis.

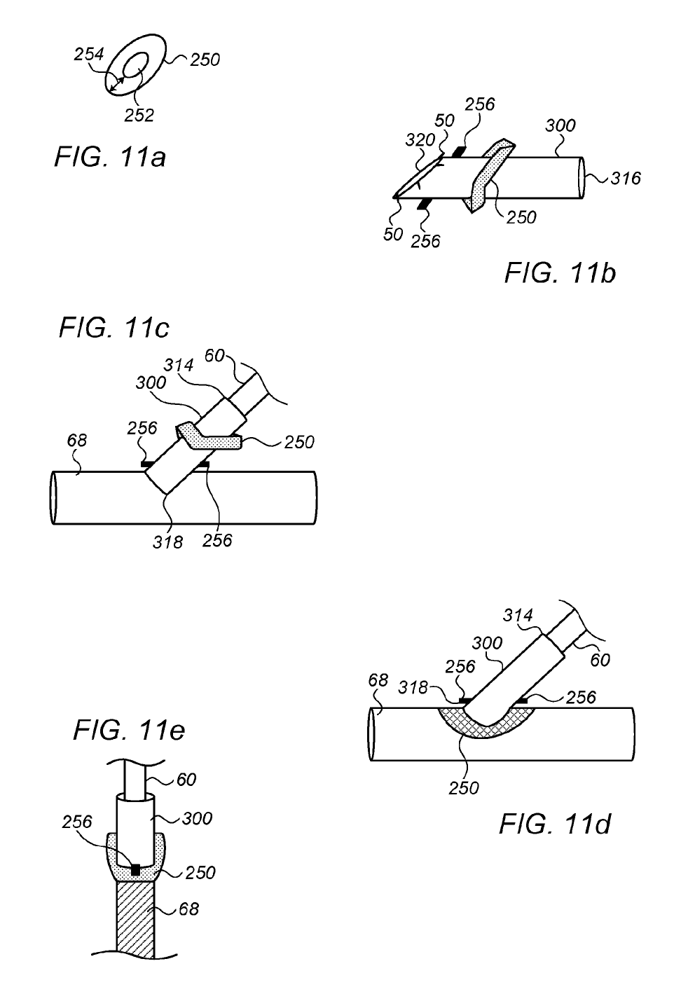

For descriptive purposes, which are by no means intended as limiting, the structure 60 may be referred to as a blood vessel as described for FIG. 4a and FIG. 4b. In one non-limiting example the second structure 68 to be joined to the blood vessel 60 inserted in the cavity of the device may be an artery. The device may be used for an end to end connection or an end to side connection of the affixed biological structure inserted and retained in the cavity of the device. FIG. 8b shows schematically an example of an end to end connection of a second structure 68 to the biological structure 60, which is retained in the device 300 and has been fixed to the device 300 described in FIG. 8a. The proximal opening 320 of the device with the end 62 of the retained biological structure 60 affixed by protrusions 50 may be inserted into the opening 70 of the second structure 68. The exposed end 72 of the second structure 68 may be fixed by the same protrusions 50 which have the end 62 of the biological structure 60 affixed to them so that the extremity 72 of the second structure 68 overlays the end 62 of the biological structure 60 held underneath. The protrusions 50 are sized in order to pierce and strongly fix to both the first structure 60 and the second structure 68. FIG. 8c shows schematically an example of an end to side connection of a biological structure 60, such as a graft 60 retained in and affixed to an exemplary device, such as shown in FIG. 2a, connected to a second structure 68 such as an artery 68. The proximal opening 320 of the device with the biological structure 60 held and retained in the cavity of the device 300 and the end 62 of the biological structure 60 (shown as dotted line) everted and fixed to the protrusions 50 about the proximal opening of the device 300 may be inserted into an opening 110 on the side of the second structure 68, such as an artery 68. The device 300 may be inserted minimally such that the end of the tissue 112 surrounding the opening 110 of the second structure 68, may be fixed to the protrusions 50 of the device to overly the end 62 of the biological structure 60 fixed by the protrusions 50 and to join the opening of the biological structure 60 held in the device 300 to the opening 110 of the second structure 68 such as the artery 68. In some embodiments, a mechanism to prevent the device 300 from displacement deeper into the biological structure 68 may be used in order to avert blockage of the biological structure 68 by the displaced device 300. Any suitable mechanism may be used. In some embodiments a restraining ring applied around the device 300 may be used in combination with a stop member fixed on the device 300 and adjacent to the proximal opening 320. An exemplary mechanism is shown schematically in FIGS. 11a-11d and described hereinafter.

FIG. 8d shows schematically an alternative non-limiting example of an end to side connection of a biological structure 60, such as a graft 60 to a second structure 68, which has a blockage, such as an artery 68, using an exemplary device 300, with a proximal opening cut at ninety degrees to the longitudinal axis, such as shown in FIG. 2a. The biological structure 60 may be affixed to the device 300 as described in FIG. 8c. The proximal opening 320 of the device 300 with the end 62 of the held biological structure 60 everted and fixed to the protrusions 50 about the proximal opening 320 of the device 300 may be inserted into an opening 110 on the side of the second structure 68, such as an artery 68. The flexibility of the second structure 68 and the positioning of the opening 110 facilitates manipulating part of the length of the second structure 68 about the opening 110 to provide a longitudinal part of the second structure spaced apart from the blockage, with the opening 110 at an acute angle to the longitudinal part of the second structure 68. The device 300 may be inserted at an angle into the angled opening 110 of the second structure 68. In some non-limiting examples the device may be inserted into the opening 110 at an angle of substantially about one hundred and eighty degrees to the longitudinal part of the second structure 68. The longitudinal axis 312 of the device 300, which is held in the lumen of the second structure 68, is parallel and in line with the longitudinal axis of at least part of the second structure 68 and is fully encircled by the second structure 68. The device 300 may be positioned such that it is substantially concentric to the second structure 68. The device 300 may include a plurality of protrusions to fix the biological structure 60 and the second structure 68 to the device 300. The plurality of protrusions 50 may be positioned on the device 300 in such a way as to prevent contact with flowing blood. In some embodiments, the protrusions 50 are positioned on the external body of the device 300 at a suitable distance from the proximal opening 320 of the device 300 in order to prevent contact with flowing blood after insertion of the device 300 into the lumen of the second structure 68. The device 300 may be inserted such that the circumference of the lumen of the second structure 68, may be fixed to the protrusions 50 of the device to overly the end 62 of the biological structure 60 fixed by the protrusions 50 and to join the opening of the biological structure 60 held in the device 300 to the second structure 68 via the opening 110 of the second structure 68, such as the artery 68. A similar method of end to side connection of a blocked structure may be used with a braided device such as shown in FIG. 1a.

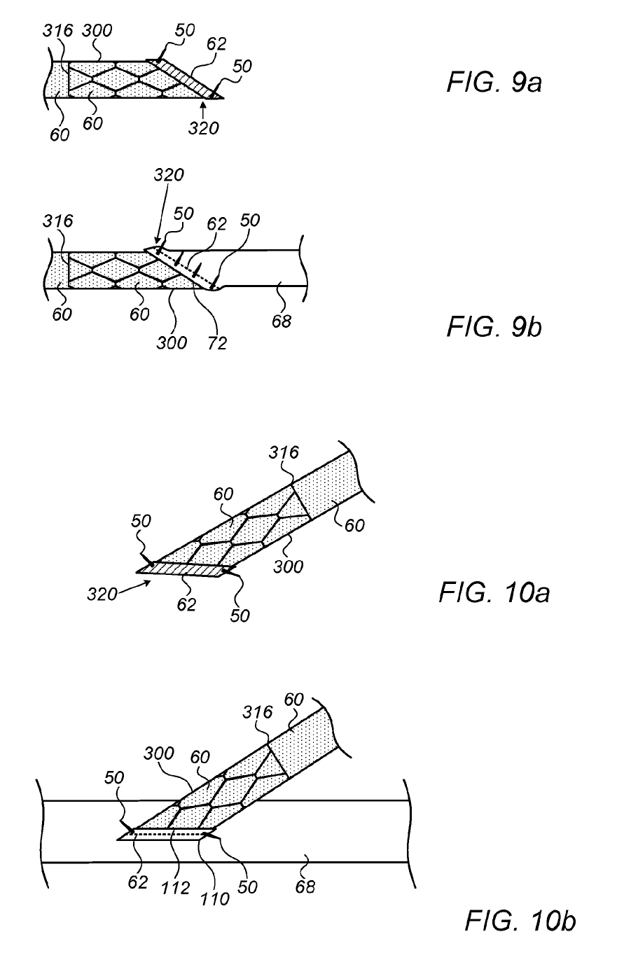

A device of the present invention may be used for an angled join, which may be useful in an end to end or an end to side anastomosis connection. An angled join may be achieved using a device of the present invention with a proximal opening cut at an angle, such as at an acute angle to the longitudinal axis of the device. FIG. 9a shows schematically an embodiment of a tubular net-like device 300 used in an end to end connection. The device may be a device 300 as described for FIG. 2b. A biological structure 60, such as, but not limited to a blood vessel 60 may be inserted through a distal opening 316 formed at ninety degrees to the longitudinal axis and into the cavity of the device 300. The end 62 of the blood vessel 60 to be joined may be everted over the angled proximal opening 320 and fixed to the device 300 with the plurality of spaced apart protrusions 50 about the proximal opening 320. Insertion of the biological structure 60 into and through the cavity may be relatively facile due to the relatively linear rigidity of the device 300 and the radial expansion of the device 300 according to the size of the biological structure 60 being inserted. As shown schematically in FIG. 9b, the proximal opening 320 of the device 300 with the first biological structure 60 affixed to the protrusions 50 of the device 300, may be inserted into the exposed end 72 of a second structure 68, so that the protrusions 50 of the device 300 fix to the second structure 68 to overly and join the end 72 of the second structure 68 to the end 62 of the first biological structure 60. The exposed end 72 of the second structure 68 may be cut at a similar angle to the angle of the proximal opening 320. However, whatever the angle of cut is of the exposed end 72 of the second structure 68, due to the arrangement and configuration of the protrusions, which conform to the shape and angle of the proximal opening, the second structure 68 may be affixed to result in the optimal angle for joining.

FIG. 10a shows a schematic view of an exemplary device 300 with an acute angled proximal opening 320 used in an exemplary end to side connection. The device 300 may feature a tubular net-like configuration as described herein for FIG. 2b. A biological structure 60, such as, but not limited to a blood vessel 60 may be inserted through a distal opening 316 cut at ninety degrees to the longitudinal axis and into the cavity of the device 300. The radius of the tubular net may expand according to the radius and size of the tubular biological structure 60 inserted into the device 300. The blood vessel 60 may be inserted until the end 62 protrudes from the proximal opening 320 of the device 300. The end 62 of the blood vessel 60 to be joined may be everted over the angled proximal opening 320 and fixed to the device 300 with the plurality of spaced apart protrusions 50 positioned about the exterior surface of the proximal opening 320. The acute angle of the proximal opening 320 and the placement of the protrusions 50 facilitates the correct angle for joining the inserted biological structure 60 to a second structure in an end to side connection such as in a bypass type connection. The affixed end 62 may adopt the dimensions, shape and angle of the proximal opening 320. As shown schematically in FIG. 10b, the proximal opening 320 of the device 300 with the held first biological structure 60 affixed to the device 300 by the protrusions 50 may be joined to the side of a second structure 68. The proximal opening 320 of the device 300 with the attached biological structure 60 may be inserted into the side of a second structure 68, such as an opening 110 in the side of an artery 68. The proximal opening 320 of the device 300 may be inserted to a minimal depth in order that the protrusions 50 on the device 300 pierce, or hold in any suitable way the end of the tissue 112 surrounding the opening 110 in the artery 68. The protrusions 50 of the device 300 fix the end of the tissue 112 surrounding the opening 110 in the second structure 68, such as an artery 68 to overly and join the opening 110 of the second structure 68 to the first biological structure 60. The connection of the first structure 60 to the opening 110 in the second structure 68 may be at an acute angle equivalent to the angle of the proximal opening 320 of the device 300. In some embodiments, a mechanism to prevent the device 300 from displacement deeper into the biological structure 68 may be used in order to avert blockage of the biological structure 68 by the displaced device. Any suitable retraining mechanism may be used. In some embodiments a restraining ring applied around the device 300 may be used in combination with a stop member fixed on the device and adjacent to the proximal opening 320 of the device. An exemplary mechanism is shown schematically in FIGS. 11a-11d and described herein.

FIG. 11a shows a schematic view of an exemplary restraining ring 250. The restraining ring 250 may have any suitable ring shape with a central cavity 252, such as a toroidal shape. The restraining ring 250 is shaped to encircle and fit about a connector device of the present invention 10, 300, as shown schematically in FIG. 11b. The cavity 252 may be sized to fit the radial dimensions of the connector device 10, 300 and allow the restraining ring 250 to encircle the connector device 10, 300 and be freely moved longitudinally along the external surface of the body of the connector device 10, 300. The central cavity 252 of the restraining ring 250 may be sized and configured in the shape of the proximal opening 320 of the device 10, 300. A restraining ring for use with a connector device 10, 300 with an angled proximal opening, such as shown in FIG. 1c and FIG. 2b may be elliptical to adopt the same angle and shape as the proximal opening. A restraining ring for use with a device 10, 300 including a proximal opening at ninety degrees to the longitudinal axis, such as shown in FIG. 1a and FIG. 2a may have a shape and angle to correspond respectively to the right angled proximal opening. In some embodiments, the restraining ring may be square shaped, or non-circular shaped. The restraining ring 250 may be made from any suitable material. In some embodiments, the ring 250 may be made from nitinol. The restraining ring may have a net-like design as described for the device shown in FIG. 2a and FIG. 2b. The restraining ring 250 may be flat, or may be thick. The width 254 of the formed part of the restraining ring 250 may be configured to correspond with at least one stop member 256, which is fixed on the connector device 10, 300 in order that the combination of the restraining ring 250 and the stop member 256 can provide a restraining mechanism to prevent displacement of the device 10, 300. The device 300 may include a plurality of stop members 256. The at least one stop members 256 may be unidirectional to allow an encircling restraining ring 250 to traverse the at least one unidirectional stop member in a direction towards the proximal end of the device 10, 300, but which prevents the encircling restraining ring 250 from moving back over the at least one unidirectional stop member 256 in a direction towards the distal end of the device 10, 300.

FIG. 11c shows a schematic view of a connector device 300 with an acute angled proximal opening, such as in FIG. 2b. The device 300 may be used to connect a first biological structure 60 and a second biological structure 68 as shown and described in FIG. 10a and FIG. 10b, which to avoid repetition will not be described again. The device 300 shown in FIG. 11c includes a restraining mechanism featuring a restraining ring 250, which encircles the device 300 and a plurality of stop members 256 which are affixed to the external surface of the tubular member of the device 300. The plurality of stop members 256 may be positioned adjacent to the proximal opening 320 and projecting out from the external surface of the device 300. FIG. 11c shows the restraining ring 250 in an initial position, which is spaced apart from the stop member 256. When the device 300 with the everted biological structure 60 is attached by the plurality of protrusions 50 to the tissue surrounding the opening in the side of the second structure 68 as described in FIG. 10b, the restraining mechanism may be reconfigured to restrain the device 300. The restraining ring 250 encircling the device 300 may be moved, such as, but not limited to sliding it, along the external surface of the device 300 from the initial position along the longitudinal length of the device 300 towards the proximal end 318 of the device 300. The restraining ring 250 may be moved down towards the proximal end 318 of the device and may traverse the plurality of stop members 256. The plurality of stop members 256 are unidirectional stop members 256 allowing the restraining ring 250 to move over the stop member 256 towards the proximal end of the device 300, but once the restraining ring 250 has passed over the stop member 256, the stop member 256 is configured to not allow the restraining ring 250 to move back in the other direction over the stop member 256 and towards the distal end 314 of the device 300. As shown in FIG. 11d, the device 300 cannot be displaced deeper into the second structure 68, such as an artery 68, as the movement of the device 300 in a direction further into the second structure 68 is blocked by the unidirectional stop members contacting the restraining ring 250. When the device 300 is in place and the restraining mechanism applied, the restraining ring 250 may sit on the second structure 68, such as an artery, or may be in close proximity to the opening in the side of the second structure 68. FIG. 11e shows a different schematic view of the arrangement of the restraining mechanism components 250, 256, wherein the restraining ring 250 is physically obstructing the stop member 256 from any movement into the lumen of the second structure 68, and consequently preventing any movement of the device 300 to which the stop member 256 is affixed, deeper into the lumen of the second structure 68.

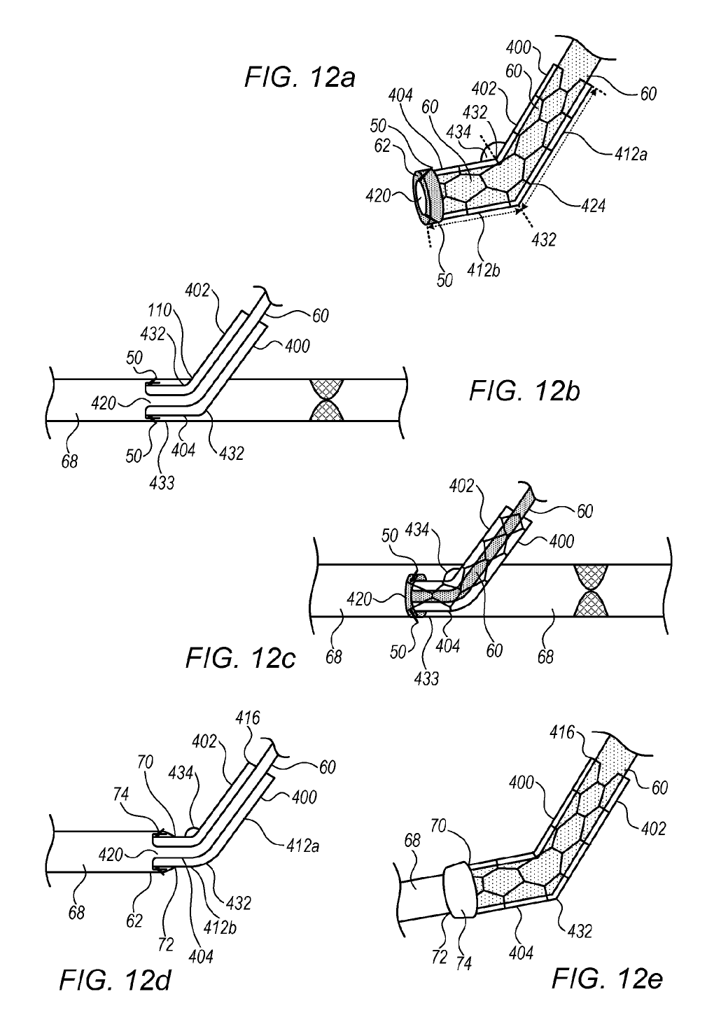

FIG. 12a shows a schematic view of another embodiment of the present invention featuring an exemplary device 400 including a body 424 with a bend, such that a first part 402 of the body 424 is at an angle to the second part 404 of the body. The device with an angled body 400 may be used to connect a biological structure 60 to a second structure 68 in an exemplary end to side connection or end to end connection. The device may feature a tubular net-like configuration as described herein for FIG. 2g. FIG. 12a shows schematically a biological structure 60 held in the cavity of the device 400 and attached to the device by a plurality of protrusions 50. A biological structure 60, such as, but not limited to a blood vessel 60 may be inserted through a distal opening 416 cut at ninety degrees to the longitudinal axis 412a and into the cavity of the device 400. The radius of the tubular net may expand according to the radius and size of the tubular biological structure 60 inserted into the device 400. The blood vessel 60 may be inserted through the device 400, through the first part 402 of the device and the second part 404 of the device until the end 62 of the blood vessel protrudes from the proximal opening 420 of the device 400. The blood vessel 60 may bend at the join 432 between the first part 402 of the device and the second part 404 of the device according to the angle of the bend of the device 400. The end 62 of the blood vessel 60 to be joined may be everted over the angled proximal opening 420 and fixed to the device 400 with the plurality of spaced apart protrusions 50 positioned about the exterior surface of the proximal opening 420. The affixed end 62 may adopt the dimensions and shape of the proximal opening 420.

As shown schematically in FIG. 12b and FIG. 12c, the proximal opening 420 of the device 400 with the first biological structure 60 affixed to it by protrusions 50 on the device 400 may be joined to the side of a second structure 68. The second structure 68 may be a structure 68 with a blockage. The second structure 68 may be any suitable structure 68. In one non-limiting example the second structure 68 may be an artery 68 with a blockage. FIG. 12b shows a cross sectional view of the end to side connection. FIG. 12c shows a view with the device in more detail. The second section of the device 404 or at least part thereof may be configured for being retained in the lumen of a second structure 68, the second structure 68 is to be joined to the first biological structure 60 held in the cavity of the device 400. Referring to both FIG. 12b and FIG. 12c, the proximal opening 420 of the device 400 with the attached biological structure 60 and the second part of the device 404 may be inserted into the side of a second structure 68, such as through an opening 110 in the side of an artery 68. The second part 404 of the device featuring the second longitudinal axis 412b and adjacent to the proximal opening 420 of the device may be aligned with the longitudinal axis of the second structure 68, in which the second part 404 of the device 400 is held, such that it is substantially concentric with the overlying second structure 68. The device 400 may be sized and the protrusions 50 positioned on the device 400 such that the protrusions 50 affixed to the end 62 of the biological structure 60, pierce, or hold in any suitable way the tissue 433 surrounding the lumen in the second structure 68 to overly the end 62 of the biological structure 60 and to attach the second structure 68 to the external surface of the device 400. The angled device 400 is configured, such that the first part 402 of the device 400 protrudes out of the opening 110 in the side of the second structure 68. The angle 434 of the first part 402 of the device 400 in relation to the second part 404 of the device 400 provides the biological structure held in the first part 402 of the device at a corresponding angle to the second structure 68 to which it is joined.

In some embodiments, the device shown in FIG. 12a with the affixed biological structure 60 may be used to join the biological structure 60 to the end of a second structure 68 in an end to end connection as shown schematically in FIG. 12d and FIG. 12e. The second structure 68 may be any suitable structure 68. In one non-limiting example the second structure 68 may be a blood vessel. FIG. 12d shows a cross sectional view of the end to end connection. FIG. 12e shows a schematic view of the end to end connection with the device in more detail.

Referring to both FIG. 12d and FIG. 12e, the proximal opening 420 of the device 400 with the attached biological structure 60 and at least a section of the adjacent second part of the device 404 may be inserted into an opening 70 at an extremity 72 of the second structure 68. The second part 404 of the device 400 featuring the second longitudinal axis 412b and adjacent to the proximal opening 420 of the device may be aligned with the longitudinal axis of the second structure 68, in which the proximal opening 420 and at least a portion of the adjacent second part 404 of the device is held, such that it is substantially concentric with the overlying second structure 68. The device 400 may be sized and the protrusions 50 positioned on the device 400 such that the protrusions 50 affixed to the end 62 of the biological structure 60, pierce, or hold in any suitable way the tissue 74 adjacent to the opening 70 of the second structure 68 such as tissue surrounding the lumen of the second structure 68, to overly the end 62 of the biological structure 60. The angled device 400 is configured, such that the proximal opening 420 and the second part 404 of the device is substantially in line with the same longitudinal axis as the second structure 68 and the first part 402 of the device 400 protrudes at the angle 434 corresponding to the bend in the device 400. The angle 434 of the first part 402 of the device 400 in relation to the second part 404 of the device 400, may provide the biological structure 60 held in the first part 402 of the device at an angle to the second structure 68 to which it is joined.

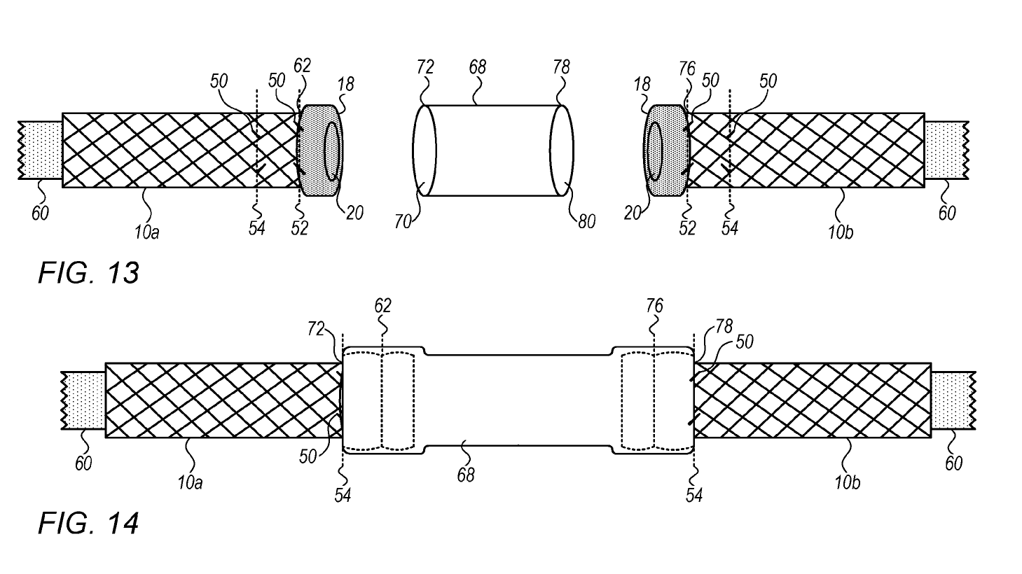

A plurality of devices of the present invention may be used for a plurality of connections of a plurality of biological and non-biological structures. The device of the present invention may be for connecting more than two biological or non-biological structures. Two devices of the present invention may be used to join a biological or a non-biological structure to two openings or two severed ends of a second structure, or to a plurality of other structures. FIG. 13 shows a schematic view of two exemplary connector devices for connecting a plurality of structures according to an aspect of the present invention. One end 62 of a biological or a non-biological structure 60 may be inserted in a first device 10a of the present invention, such that the severed end 62 of the inserted structure is everted and fixed to a first line 52 of a plurality of protrusions 50 adjacent to the proximal opening 20 of the first device 10a as similarly described hereinabove in FIG. 5. A second end 76 of the biological or non-biological structure 60, which may be the second end of the same biological or non-biological structure 60 as inserted in the first device 10a, or which may be an end of an additional biological or non-biological structure 60 may be inserted in a second device 10b of the present invention, such that the severed end 76 of the inserted structure is everted and fixed to a first line 52 of a plurality of protrusions 50 adjacent to the proximal opening 20 of the second device 10b as shown schematically in FIG. 13. A structure 68, which is to be joined to the biological or non-biological structures 60 inserted in the first device 10a and the second device 10b of the present invention is brought into contact so that a first opening 70 of the structure 68 is in line with the proximal opening 20 of the first device 10a and the second opening 80 of the structure 68 is in line with the proximal opening 20 of the second device 10b.

FIG. 14 shows a schematic view of two exemplary connector devices 10a, 10b connecting a plurality of structures 60, 68, 60 according to an aspect of the present invention. As shown schematically in FIG. 14 the first end 72 of the structure 68 is fixed to the first device 10a and overlays the everted end 62 (shown with a dotted line as it is covered) of the biological or non-biological structure 60 accommodated in the first device 10a and the second end 78 of the structure 68 is fixed to the second device 10b and overlays the everted end 76 (shown with a dotted line as it is covered) of the biological or non-biological structure 60 accommodated therein. Referring to both FIG. 13 and FIG. 14, in a non-limiting example, the first device 10a with the everted end 62 of the biological or non-biological structure 60 accommodated therein may be inserted into the first opening 70 of the structure 68, until the first end 72 of the structure 68 contacts the protrusions 50 on the external surface of the first device 10a. In an alternative method the first end 72 of the structure 68 may be pulled or manipulated in any suitable way over the proximal opening of the first device 10a and over the everted end of the biological or non-biological structure 60 accommodated therein until the first end 72 of the structure 68 contacts the protrusions 50 on the external surface of the first device 10a. The protrusions may fix the first end 72 of the structure 68 to the first device 10a so that part of the structure 68 overlays the everted end of the biological or non-biological structure 60 accommodated in the first device 10a. Similarly, the second device 10b with the everted end 76 of the biological or non-biological structure 60 accommodated therein may be inserted into the second opening 80 of the structure 68, until the second end 78 of the structure 68 contacts the protrusions 50 disposed on the second device 10b. In an alternative embodiment the second end 78 of the structure 68 may be pulled or manipulated in any suitable way over the proximal opening of the second device 10b and over the everted end of the biological or non-biological structure 60 accommodated therein until the second end 78 of the structure 68 contacts the protrusions 50 on the second device 10b.

The protrusions 50 may fix the second end 78 of the structure 68 to the second device 10b so that part of the structure 68 overlays the everted end of the biological or non-biological structure 60 accommodated in the second device 10b. As shown in FIG. 14, in this way the structure 68 is joined to each of two ends of the biological or non-biological structure/s 60, wherein each end of the biological or non-biological structure/s 60 is everted over a device 10a, 10b of the present invention and connects the two ends of the biological or non-biological structure/s 60 to create a continuous lumen, through which fluid, such as body fluids may flow.

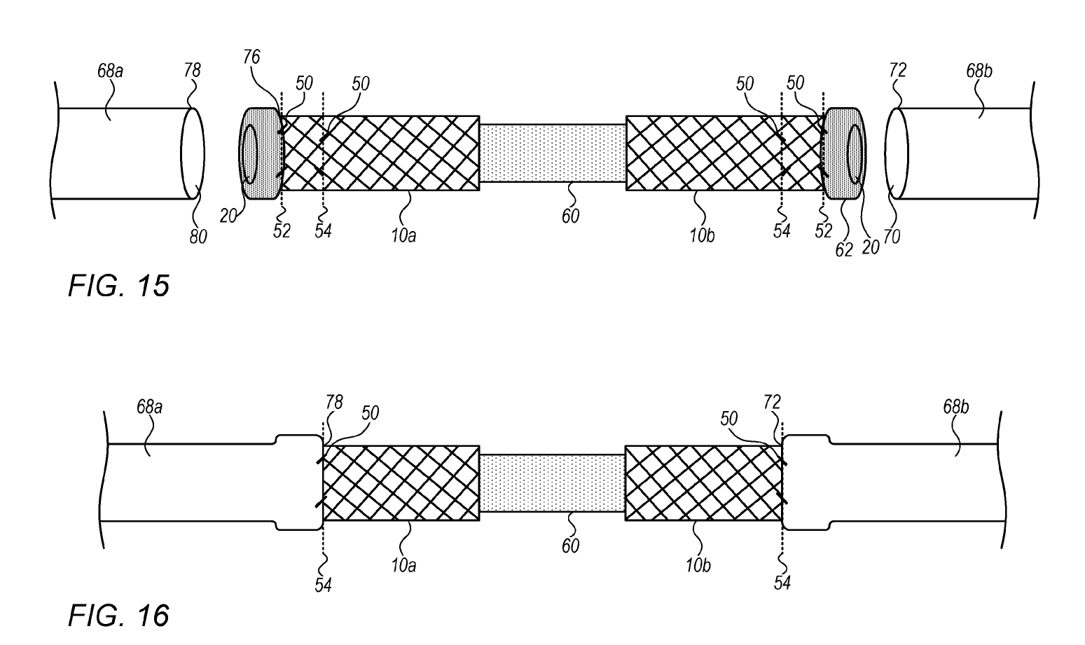

FIG. 15 and FIG. 16 show schematically a further example of how a plurality of exemplary devices of the present invention may be used to connect a plurality of biological and/or non-biological structures. FIG. 15 shows two devices 10a, 10b of the present invention, which are applied to the two ends 62, 76 of a length of a structure 60, such as, but not limited to a blood vessel 60. The end 62 of the structure 60 is inserted through the cavity of the device 10a and everted over the edge of the opening 20 of the device 10a. Similarly, the end 76 of the structure 60 is inserted through the cavity of the device 10b and everted over the edge of the opening 20 of the device 10b. The everted ends 62 and 76 may be connected to a second biological structure 68, which may be two parts 68a, 68b of the same structure 68 or two different structures 68a, 68b. As shown schematically in FIG. 16, the exposed ends 78, 72 of the structures 68a, 68b may be connected to the everted ends of the structure 60 to create a continuous lumen, from structure 68a through structure 60 and to structure 68b, through which fluid, such as body fluids may flow. The exposed end 78 of structure 68a may be connected to the everted end 76 of structure 60 in a similar way as described in FIG. 14. The exposed end 72 of structure 68b may be connected to the everted end 62 of structure 60 in a similar way as described in FIG. 14. In one non-limiting method, the respective exposed ends, 78, 72 of both structures 68a, 68b may be pulled over the respective everted end 76, 62 of structure 60 and over the device 10 in which structure 60 is held and retained. In an alternative non-limiting method, the connection may be made, by inserting both devices 10a, 10b with the affixed respective everted ends 76, 62 of structure 60 into the exposed opening 80, 70 of the respective structure 68a, 68b. The ends of structures 68a, 68b may be fixed to the respective device 10a, 10b to encircle and overlay the respective everted part 76, 62 of structure 60, the fixing facilitated by protrusions 50 disposed on the external surface of the device 10.

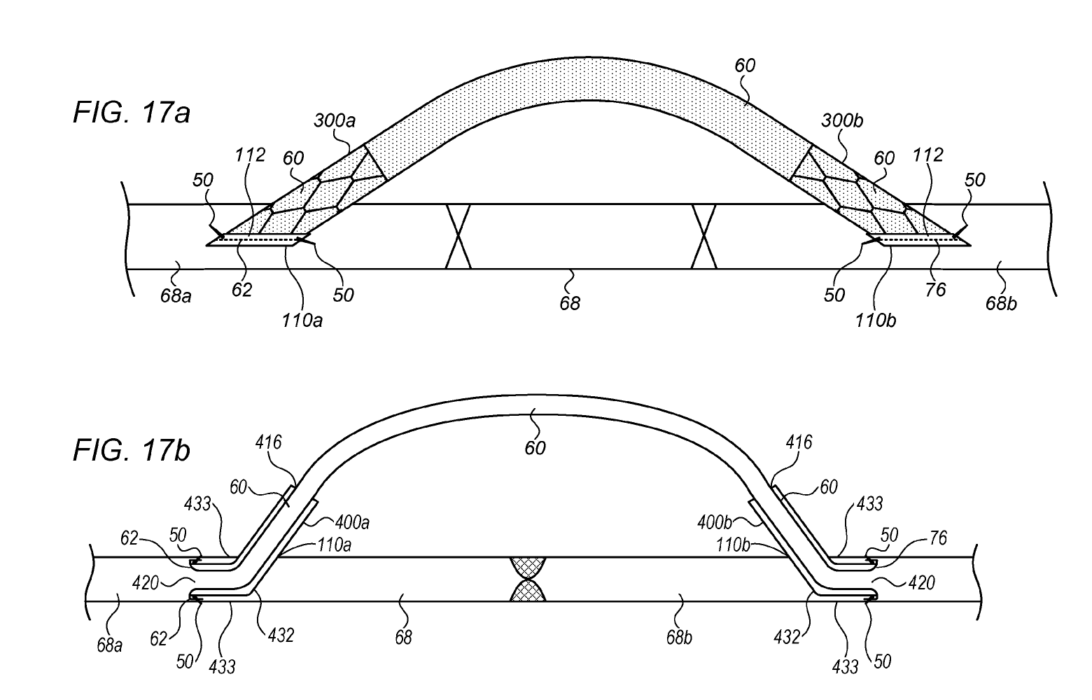

FIG. 17a shows schematically a plurality of exemplary net-like devices 300 with proximal openings cut at an acute angle to the longitudinal axis of the present invention, the devices 300 connected to a plurality of biological and/or non-biological structures. FIG. 17a shows two devices 300a, 300b of the present invention for connecting a plurality of structures. The devices 300a, 300b may include restraining mechanisms as shown in FIGS. 11a-11e and described hereinbefore. The structures may be biological and non-biological structures. In one non-limiting example, the devices 300a, 300b, may be used in a bypass connection to connect a graft 60 to an artery 68 at a position 68a before a blockage and a position 68b after a blockage. FIG. 17a may describe a system for replacement of and/or augmenting suturing in a bypass procedure. In the description below, although one structure 60 may be referred to as a graft and a second structure 68 may be referred to as an artery, this is intended to be non-limiting and the devices 300a, 300b can be used to join other structures in different procedures. Two devices 300a, 300b may be applied to the ends of a length of a structure 60. Each device may be applied to a respective end 62, 76 of the structure 60 which will be referred to for descriptive purposes as a graft 60, but which is not limiting. The devices 300a, 300b may feature a net-like configuration as described herein and shown for example in FIG. 2b. The devices 300a, 300b, may include an acute angled proximal opening 320 as described hereinabove. The first end 62 of the graft 60 may be inserted through the cavity of the first device 300a and everted over the edge of the proximal opening 320 of the device 300a. Insertion of the first end of the graft into the device may cause the device 300a to radially expand to fit the radius of the graft. The proximal opening 320 of the device may remain unchanged in size and the everted first end 62 of the graft 60 may adopt the shape and size of the proximal opening 320 of the device 300a. The end 62 of the structure 60 may be fixed by protrusions 50 positioned on the proximal opening of the device 300a, which may fix the end 62 of the blood vessel graft 60 to the external surface of the device 300a, exposing the opening to be joined of the first extremity 62 of the blood vessel 60. Similarly, the second end 76 of the graft 60 may be inserted through the cavity of the second device 300b and everted over the edge of the proximal opening 320 of the second device 300b. Insertion of the second end 76 of the graft 60 into the device 300b may cause the device 300b to radially expand to fit the radius of the graft 60. The proximal opening 320 of the device 300b may remain unchanged in size and the everted second end 76 of the graft 60 may adopt the shape and size of the proximal opening 320 of the device 300b. The second end 76 of the graft 60 may be fixed by protrusions 50 positioned on the proximal opening of the device 300b, which may fix the second end 76 of the blood vessel 60 to the external surface of the device 300b, exposing the opening to be joined of the second extremity 76 of the blood vessel 60. The everted ends 62 and 76 of the graft 60 may be connected to a second biological structure 68, each end 62, 76 may be connected to a respective different position or part 68a, 68b of the second biological structure 68. In some embodiments, the second biological structure 68 may be an artery 68 and the first structure 60 may be a graft 60 to be connected to two spaced apart openings 110a, 110b, of the artery 68, such as in a bypass connection. One opening 110a may be cut or positioned in a first part of the artery 68a and the second opening 110b may be cut or positioned in a second part of the artery 68b. The devices 300a, 300b of the present invention may be used to connect the openings 110a, 110b in the artery 68 to the everted ends 62, 76 of the graft 60 to create a continuous lumen, from the first part of the artery 68a through the graft structure 60 and to the second part of the artery 68b, through which fluid, such as body fluids may flow. The opening 110a of the first part of the artery 68a may be connected to the everted first end 62 of the graft 60. The opening 110b of the second part of the artery 68b may be connected to the everted second end 76 of the graft 60. The connecting may be done by inserting device 300a with the affixed everted first end 62 of the graft 60 into the opening 110a of the artery 68. The device 300a may be inserted to a minimal depth for fixing the surrounding tissue 112 of the opening 110a in the artery to the protrusions 50 on the device 300a to overlay and connect the first opening 110a of the artery to the first end 62 of the graft structure 60. Similarly, the device 300b with the affixed everted second end 76 of the graft structure 60 may be inserted into the second opening 110b of the artery 68. The device 300b may be inserted to a minimal depth for fixing the tissue 112 surrounding the opening 110b of the artery 68 to the protrusions 50 on the device 300b to overlay and connect the second opening 110b of the artery 68 to the second end 76 of the graft structure 60. In an embodiment, wherein the devices 300a, 300b include a restraining mechanism, the restraining mechanism can be applied as described previously in FIGS. 11a-11e to prevent displacement of the devices 300a, 300b further into the lumen of the artery 68. The devices 300a, 300b may connect each end of the graft 60 to the respective opening in the artery at an angle equivalent to the angle of the proximal opening 320 of the device 300a, 300b. In some embodiments, such as described in FIG. 17a, the angle of connection may be an acute angle. In an alternative embodiment, such as when using a device with a proximal opening formed at ninety degrees to the longitudinal axis, the connection may be at an angle of ninety degrees. The two devices 300a, 300b may promote biological joining of the connected structures 60, 68 and the devices 300a, 300b may be left in place after the tissues have joined.

Similarly, a plurality of exemplary net-like devices with angled bodies 400 of the present invention, such as shown in FIG. 2g and FIGS. 12a-12c may be used to connect a plurality of structures, such as an artery and a blood vessel graft. FIG. 17b shows a schematic cross-sectional side view of two exemplary net-like devices with angled bodes 400 of the present invention employed in a bypass type connection according to an aspect of the present invention. FIG. 17b shows a system wherein the connection devices 400 can replace and/or augment suturing in a bypass procedure. A plurality of devices 400a, 400b, may be used to connect a blood vessel graft 60 to a blocked artery 68. In order to avoid repetition, each of the two devices 400a, 400b may be as described previously in FIG. 2g. One end 62 of a graft 60 may be inserted into the first device 400a and the end 62 affixed to the device 400a as shown in FIG. 12a and described herein. A second extremity 76 of the graft 60 may be inserted into the second device 400b and the end 76 affixed to the device 400b as shown in FIG. 12a and described herein. The everted ends 62 and 76 of the graft 60 may be connected to the artery 68, each end 62, 76 may be connected to a respective different part 68a, 68b of the artery 68. The first device, such as device 400a, may be inserted into a first opening 110a of an artery 68 before a blockage as shown and described in FIG. 12b and FIG. 12c and the second device, such as device 400b may be inserted into a second opening 100b of an artery 68 after a blockage as shown and described in FIG. 12b and FIG. 12c. The devices 400a, 400b of the present invention may be used to connect the artery 68 before a blockage 68a and after a blockage 68b via openings 110a, 110b in the artery 68 to the everted ends 62, 76 of the graft 60 to create a continuous lumen, from the first part of the artery 68a through the graft structure 60 and to the second part of the artery 68b, through which fluid, such as body fluids may flow. The angled devices 400a, 400b may facilitate connection of the graft 60 and angling the length of the graft 60 according to the angle of the bend in the device away from the longitudinal axis of the artery 68.

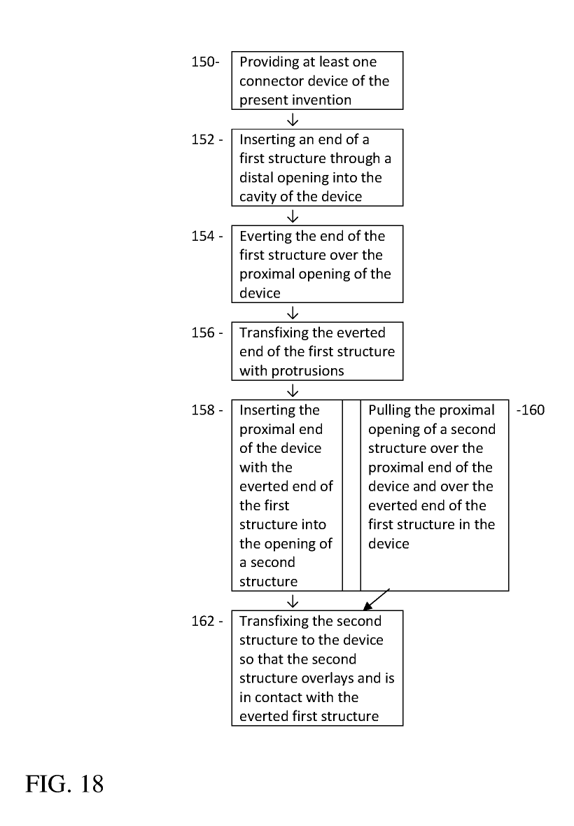

FIG. 18 shows a flow chart of an exemplary method of using the connector device according to an aspect of the present invention. FIG. 18 shows a method of connecting the end of a first structure to the end of a second structure. The first structure and the second structure may be two severed ends of the same structure, or ends of different structures. The first structure may be a biological structure or a non-biological structure and the second structure may be a biological structure or a non-biological structure. In one non-limiting example the first structure may be a blood vessel and the second structure may be an artery.

A user may perform any procedure necessary prior to, during and after use of the device of the present invention. In one non-limiting example, wherein a blood vessel has been cut, a clamp or a plurality of clamps or any equivalent may be applied at a suitable position of the severed blood vessel to temporarily stop or reduce blood flow. A user may provide a device of the present invention and may select a device with dimensions suitable for optimally accommodating the structures to be inserted and joined 150. A user may manipulate the device. In an example of a tubular braid device manipulation may include pushing the ends of the device in order to expand the diameter of the device for more facile insertion of the structure to be accommodated. In an example of a net-like device, a user may not need to manipulate the device, but may cause radial expansion of the device by the internal radial force of the structure during insertion. A user may insert one severed end of the first structure through a distal opening of a connector device of the present invention and into the cavity of the connector device 152. A user may insert the severed end of the first structure until it is adjacent to, or is protruding from the proximal opening of the device. A user may evert the end of the first structure over the edge of the proximal opening of the device 154. The opening of the severed everted end of the first structure may adopt the shape of the proximal opening of the device. In an example wherein the proximal opening of the device is an acute angled cut opening, the shape and size of the severed exposed everted end of the first structure will adopt substantially the same acute angled shape. An acute angled proximal opening of the device will result in a larger exposed opening of the structure to be joined, which may enable greater blood flow, greater surface area for the join and an easier procedure for the user. A user may fix the everted end of the first structure with protrusions disposed on the external surface of the device adjacent to or on the proximal opening of the device 156. The protrusions may be spikes, which may pierce and/or hook the tissue of the everted end. A user may connect a second structure to the first structure in any suitable way. In one method a user may insert the proximal end of the device with the everted end of the structure accommodated therein into the opening of a second structure, the second structure to be joined to the first structure 158. A user may insert the proximal end of the device until the second structure is in contact with protrusions on the external surface of the device. In an alternative method a user may pull the proximal opening of the second structure over the proximal end of the device and over the everted end of the first structure held in and by the device 160. A user may pull the end of the second structure over the device until the second structure is in contact with protrusions on the external surface of the device. The protrusions may pierce and/or hook the tissue adjacent to the opening of the second structure and fix the second structure to the device 162. The protrusions for fixing the second structure may be the same protrusions used to fix the first structure. Protrusions configured for fixing both the first structure and the second structure may be sized for piercing and/or hooking both structures. The protrusions for fixing the second structure may be a second separate and spaced apart line of protrusions from the protrusions for fixing the first structure and may be configured for fixing the second structure. The second structure may be fixed to the device, such that it overlays and contacts the everted end of the first structure accommodated in the device. The overlaid and contacted everted portion of the first structure may join with the overlying portion of the second structure. The join may be substantially sealed, preventing leaks and facilitating healing. In an example, wherein the device includes a restraining mechanism, the restraining mechanism can be applied as described previously in FIGS. 11a-11e to prevent displacement of the device further into the lumen of the second structure. As a result of the properties of the device, the device may adopt the flexibility of the first structure accommodated therein and the second structure fixed thereon. As such, the two joined structures may heal, to form a flexible, non-restricted and continuous connected structure, which may be a tubular hollow structure with a continuous lumen though which fluids, such as body fluids may flow. The order of the steps of the method is not meant to be limiting and any suitable order may be used.

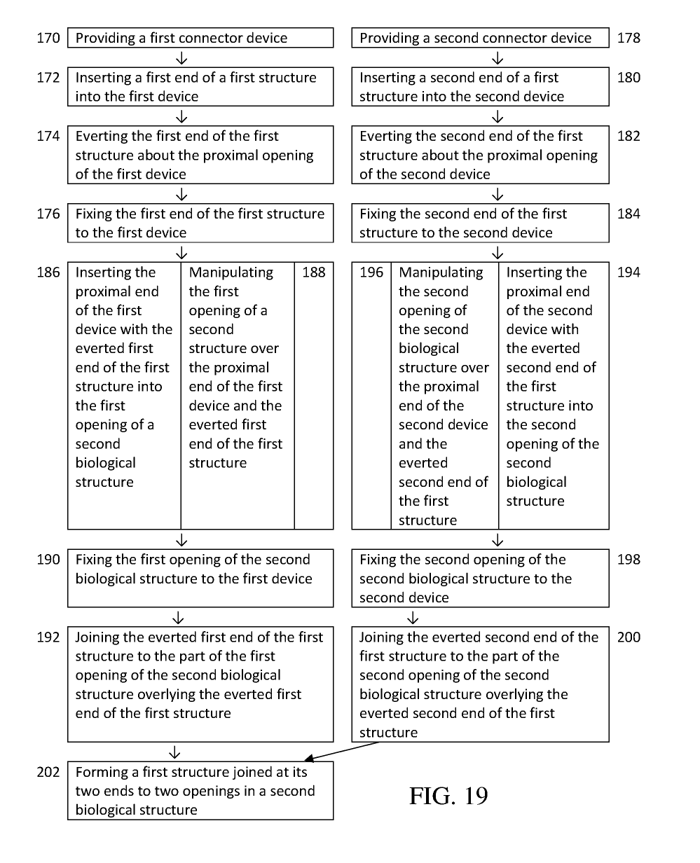

FIG. 19 shows a flow chart of an exemplary method of using a plurality of connector devices according to an aspect of the present invention. The devices may be any suitable devices of the present invention. The devices may be the same, or a combination of devices with different configurations and or sizes. FIG. 19 shows a method of connecting a first end of a first structure and a second end of a first structure to two openings of a second structure. The first structure and the second structure may be any suitable combination of biological structures and/or non-biological structures. In one non-limiting example the first structure may be a blood vessel graft and the second structure may be an artery.

A user may perform any procedure necessary prior to, during and after use of the device of the present invention. In one non-limiting example, wherein a blood vessel has been cut, a clamp or a plurality of clamps or any equivalent may be applied at a suitable position of the severed blood vessel to temporarily stop or reduce blood flow. A user may treat a biological structure or other structure prior to using the connector device of the present invention. In one aspect, the device may include at least one composition, which may treat the tissue.

A user may provide a first device of the present invention and may select a device with dimensions suitable for optimally accommodating the structures to be inserted and joined 170. A user may manipulate the first device. In an example of using a tubular braid, manipulation may include pushing the ends of the device in order to expand the diameter of the first device for more facile insertion of the structure to be accommodated. In an example of a net-like device, a user may not need to manipulate the device, but may cause radial expansion of the device by the internal radial force of the structure during insertion. A user may insert one severed end of the graft through a distal opening of the first connector device of the present invention and into the cavity of the first connector device 172. A user may insert the severed end of the graft until it is adjacent to, or protruding from the proximal opening of the first device. A user may evert the first end of the graft over the edge of the proximal opening of the first device 174. The opening of the everted first end of the graft may adopt the dimensions, shape and angle of the proximal opening of the first device. In an example wherein the proximal opening of the first device is an opening formed at an acute angle to the longitudinal axis, the shape and size of the exposed first everted end of the graft will adopt substantially the same angled shape of the proximal opening of the device. An acute angled proximal opening of the first device will result in a larger exposed opening of the first end of the graft to be joined, which may enable greater blood flow, greater surface area for the join and an easier procedure for the user. The acute angled proximal opening may also fix the everted first end of the graft in the desired angle for optimal joining. A user may fix to the first device the everted first end of the graft with a plurality of spaced apart protrusions disposed on the external surface of the first device adjacent to or on the proximal opening of the first device 176. The protrusions may be spikes, which may pierce and/or hook the tissue of the everted end.

A user may provide a second device of the present invention and may select a device with dimensions suitable for optimally accommodating the structures to be inserted and joined 178. A user may manipulate the second device. In an example of using a tubular braid manipulation may include pushing the ends of the device in order to expand the diameter of the second device for more facile insertion of the structure to be accommodated. In an example of a net-like device, a user may not need to manipulate the device, but may cause radial expansion of the device by the internal radial force of the structure during insertion. A user may insert a second end of the graft through a distal opening of the second connector device of the present invention and into the cavity of the second connector device 180. A user may insert the second end of the graft until it is adjacent to, or protruding from the proximal opening of the second device. A user may evert the second end of the graft over the edge of the proximal opening of the second device 182. The opening of the everted second end of the graft may adopt the dimensions, shape and angle of the proximal opening of the second device. In an example wherein the proximal opening of the second device is an acute angled opening, the shape and size of the exposed everted second end of the graft will adopt substantially the same acute angled shape as the proximal opening of the device. An acute angled proximal opening of the second device will result in a larger exposed opening of the second end of the graft to be joined to the artery, which may enable greater blood flow, greater surface area for the join and an easier procedure for the user. The angled proximal opening may also fix the everted second end of the graft in the desired angle for optimal joining to an artery. A user may fix to the second device the everted second end of the graft with protrusions disposed on the external surface of the second device adjacent to or on the proximal opening of the second device 184. The protrusions may be spikes, which may pierce and/or hook the tissue of the everted end.

A user may insert the proximal end of the first device with the everted first end of the graft fixed thereon, into a first opening of an artery, to join the first end of the graft to the first opening of the artery 186. A user may insert the proximal end of the first device until the tissue surrounding the first opening of the artery is in contact with protrusions on the external surface of the first device. A user may insert the proximal end of the first device to a minimal depth into the first opening of the artery for contacting and fixing the tissue surrounding the first opening to the protrusions on the first device. In an example wherein the first device is a device featuring a body with a bend, such as shown in FIG. 2d, a user may insert the proximal end of the first device into the opening of the artery and may insert the device until the second part of the device is held in the artery, such that the second part of the device is substantially concentric with the circumference of the artery. In such an embodiment, the first part of the first device will protrude out of the first opening.

Alternatively, a user may manipulate the opening of an artery over the proximal end of the first device and over the portion of the first end of the graft everted over the device until the tissue surrounding the opening in the artery is in contact with protrusions on the external surface of the first device. Manipulation may include pulling the first opening of the artery over the device 188.

The protrusions may pierce and/or hook the tissue adjacent to the first opening of the artery and fix the first opening of the artery to the first device 190. In an embodiment wherein the first device is a device featuring a body with a bend, such as shown in FIG. 2d the protrusions may hook the tissue of the artery surrounding the lumen, which is adjacent to the protrusions. The protrusions for fixing the first opening of the artery may be the same protrusions used to fix the first end of the graft to the device. Protrusions configured for fixing both the first end of the graft and the first opening of the artery may be sized for piercing and/or hooking both structures. The protrusions for fixing the first opening in the artery may be a second separate and spaced apart line of protrusions from the protrusions for fixing the first end of the graft and may be configured for fixing the first opening in the artery. The first opening in the artery may be fixed to the first device, such that it overlays and substantially directly contacts the everted first end of the graft accommodated in the first device. The overlaid and contacted everted portion of the first end of the graft may join with the overlying portion of the first opening of the artery 192. In an example, wherein the device includes a restraining mechanism, the restraining mechanism can be applied as described previously in FIGS. 11a-11e to prevent displacement of the device further into the lumen of the artery.

Similarly, a user may insert the proximal end of the second device with the everted second end of the graft fixed thereon, into a second opening of an artery, to join the second opening of the artery to the everted second end of the graft 194. A user may insert the proximal end of the second device until tissue surrounding the second opening in the artery is in contact with protrusions on the external surface of the second device. A user may insert the proximal end of the second device to a minimal depth into the second opening of the artery for contacting and fixing the tissue surrounding the second opening to the protrusions on the second device. In an example wherein the second device is a device featuring a body with a bend, such as shown in FIG. 2d, a user may insert the proximal end of the second device into the opening of the artery and may insert the device until the second part of the device is held in the artery, such that the second part of the device is substantially concentric with the circumference of the artery. In such an example, the first part of the second device will protrude out of the first opening.

Alternatively, a user may manipulate the second opening of an artery over the proximal end of the second device and over the portion of the second end of the graft everted over the second device until the tissue surrounding the second opening of the artery is in contact with protrusions on the external surface of the second device. Manipulation may include pulling the second opening of the artery over the second device 196.

The protrusions may pierce and/or hook the tissue adjacent to the second opening of the artery and fix the tissue about the second opening of the artery to the second device 198. In an embodiment wherein the second device is a device featuring a body with a bend, such as shown in FIG. 2d the protrusions may hook the tissue of the artery surrounding the lumen, which is adjacent to the protrusions. The protrusions for fixing the second opening in the artery may be the same protrusions used to fix the second end of the graft to the second device. Protrusions configured for fixing both the second end of the graft and the second opening of the artery may be sized for piercing and/or hooking both structures. In some examples, the protrusions for fixing the second opening of the artery may be a second separate and spaced apart line of protrusions from the protrusions for fixing the second end of the graft and may be configured for fixing the second opening of the artery. The second opening of the artery may be fixed to the second device, such that it overlays and substantially directly contacts the everted second end of the graft accommodated in the second device. The overlaid and contacted everted portion of the second end of the graft may join with the overlying portion of the second opening in the artery 200. In an example, wherein the device includes a restraining mechanism, the restraining mechanism can be applied as described previously in FIGS. 11a-11e to prevent displacement of the device further into the lumen of the artery. The method results in forming a graft joined at each end to respective openings in an artery in a bypass type connection 202. The joined structures may facilitate a continuous lumen through which fluids, such as body fluids can flow. Although, the method is disclosed for use in connection of a graft with openings in an artery, this is not meant to be limiting. The same method may be used to join different biological structures and non-biological structures.

The joins may be substantially sealed, preventing leaks and facilitating healing. Due to the properties of the devices described herein, the devices may adopt the flexibility of the structures accommodated therein and the structure/s fixed thereon. The joined structures may heal, to form a flexible, non-restricted and continuous connected structure, which may be a tubular hollow structure. In one non-limiting example surgical glue may be deposited in the coating or part of the coating of the internal or external surface of the device. The surgical glue may augment fixing structures to the device and/or joining of the structures. In one non-limiting example a glue may be applied about the everted end of a first structure fixed to the device before joining it to the second structure. In one non-limiting example a glue may be applied to the joined structures after joining. In an example of using a tubular braid device of the present invention, at any suitable stage, such as, but not limited to when inserting a structure into the tubular braid device a user may manipulate the device by applying pulling or pushing forces in order to expand or contract the diameter of the device for facile insertion and/or repositioning and optimal positioning of the structures. The order of the steps of the methods is not meant to be limiting and any suitable order may be used. The device/s of the present invention may remain connected to the biological or non-biological structures. In a non-limiting example the device/s may degrade after the structures have joined.

The connector device of the present invention is relatively facile for use by a user. A user may be any suitable user, such as, but not limited to a doctor, a surgeon, a nurse, a medical technician, a veterinarian, or any suitable medical professional or individual. The device may be used and a procedure using the device may be performed by more than one user. The device may be used to join a biological tissue to another biological tissue, or to join two parts of a biological tissue together, or to join a biological tissue to a non-biological structure. The biological tissues may have been cut or damaged, or may be in an unconnected state for any reason. The device may be used to join biological tissues that have been severed with any type of cut, such as but not limited to a cross-sectional cut, a straight cut, an angled cut and a combination thereof. The biological tissue/s to be joined may be located internally in the body of a patient. The connector device may be packaged in sterile packaging, which may be opened before use. The device may be inserted manually or may be inserted via suitable insertion means, which may be employed in for example keyhole surgery.

A user may apply more than one connector device of the present invention according to need. The more than one connector devices may be applied to different parts of the same biological tissue or to different biological tissues. Application of the device of the present invention may be one step or any suitable number of steps of any suitable surgical or medical procedure.

The device of the present invention may be used instead of other methods of connecting biological tissues, or in addition to such methods. In one aspect, the device of the present invention may be used in addition to suturing and may augment suturing. Such a combination of methods may be useful in a case where there is a need for reinforcement such as when there is a danger of stitches rupturing.

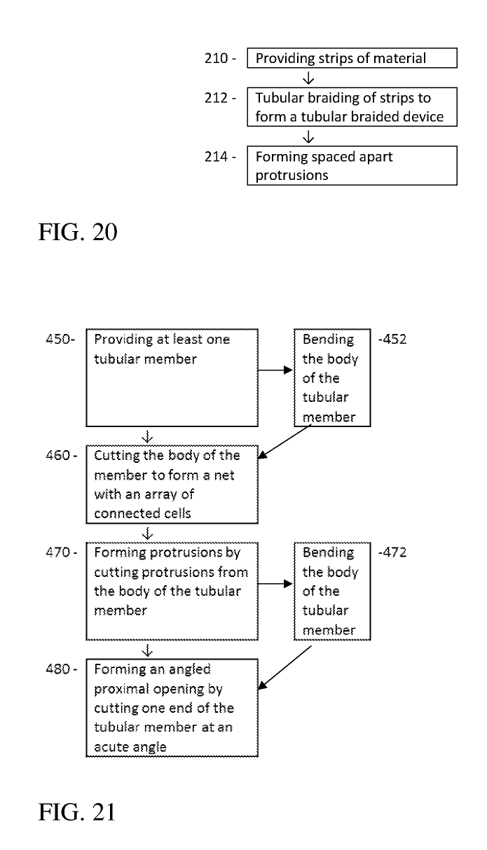

FIG. 20 shows a flow chart of an exemplary method of production of a tubular braid connector device according to an aspect of the present invention. A plurality of lengths of filaments may be provided 210. The filaments may be of any suitable material. In some embodiments the filaments are made of nitinol. The filaments may be sized and the number of filaments used may be determined according to the end use of the device. The filaments may be rounded filaments for optimal flexibility of the device. In one non-limiting example the filaments may be flat filaments, or a mixture of flat and rounded filaments. The device of the present invention may be produced by tubularly braiding the plurality of lengths of filaments to form a tubular braid 212. Protrusions, such as hooks or spikes may be formed in spaced apart relation about the circumference of the device 214. Any suitable number of protrusions may be formed. In some embodiments at least three protrusions are formed. The protrusions may be equidistant from each other. In some embodiments, some of the braided filaments are manipulated to form a hook or spike. Manipulating the braided filament may include cutting a filament length to form an end at a predetermined position about the circumference of the tubular braid and hooking the end to protrude away from the body of the tubular braid. A braided filament, which may be manipulated to form a protrusion may be a thicker filament than a filament on which a protrusion is not formed. In some embodiments, the filaments with and without the protrusions are the same size. In one non-limiting example, the filaments with the protrusions are constructed from a different material than the filaments without the protrusions. In some embodiments, a protrusion is formed by fusing a protrusion to the braided device, or gluing, or tying, or sewing, or using any other suitable method to attach a protrusion. In one non-limiting example, a filament with a protrusion is weaved into the braided device. In an embodiment, wherein the same line of protrusions is for fixing the ends of two structures to be joined, the size of the protrusion is determined to effectively pierce and hook both structures.

In some embodiments forming the protrusions may include forming two separated lines of spaced apart hooks positioned about the circumference of the tubular braid. In some embodiments one line of spaced apart protrusions is for fixing one structure to the device and the second line of spaced apart protrusions is for fixing a second structure to be joined to the first structure. The distance between the two lines of protrusions may be calculated according to the end use of the device and for facilitating optimal flexibility of the device and connected structures. In one non-limiting example the distance between the two lines of protrusions may be less than about 6 mm. An interior or exterior surface or part thereof of the tubular braid may be precoated with a material such as an adhesive, or any other suitable coating. The manufacture may be done manually or mass produced in a production line by suitable machinery. The braided ends of the device may result in an uneven edge. The ends of the device may be given a relatively smooth finish by any suitable procedure such as tucking in, or folding, or gluing, or stitching the ends of the braid so that the individual filaments or strips do not protrude or stick out in a way that can be detrimental to a biological structure. The device may be packaged in a suitable sterile packaging, which may be sealed. The order of the steps of the method is not meant to be limiting and may be in any suitable order.

FIG. 21 shows a flow chart of an exemplary method of production of a tubular net-like connector device according to an aspect of the present invention. A hollow tube of a biocompatible material with the desired thickness, length and radius may be formed or provided. A non-limiting example of a suitable material is nitinol 450.

In a non-limiting example of the production of a device featuring a body with a bend, the hollow tube may be bent 452. Methods, such as temporarily filling the tube may be used in order to maintain the radial dimensions during the bending process. The bend may be applied at a suitable position on the tube to facilitate a suitable length of a first part of the device before the bend and a suitable length of a second part of the device after the bend.

The hollow tube may be cut to form a net design. A laser may be used to cut out the desired design of the net. The laser may cut out the material, which forms the tube body, to form an array of interconnected net-like open cells of the desired dimensions and shapes 460. A plurality of hooking protrusions may be formed. The protrusions may be formed, but for example laser cut of the tubular body to form a plurality of protrusions, such as spikes 470. The cutting out of the protrusions may be done simultaneously with the cutting to form the array of connected cells of the net. The protrusions may be formed according to the design of a user. In some embodiments, the protrusions are formed spaced apart about the external surface of the proximal opening. In some embodiments, one line of protrusions are formed for fixing both structures to be joined with the device. In some embodiments, a plurality of lines of protrusions are formed, one line for fixing to one structure and the second line for fixing to a second structure to be joined to the first structure with the device.

In a second non-limiting example of the production of a device featuring a body with a bend, the bending may be done after production of the linear net-like tube 472. Methods, such as temporarily filling the tube may be used in order to maintain the radial dimensions during the bending process. The bend may be applied at a suitable position on the tube to facilitate a suitable length of a first part of the device before the bend and a suitable length of a second part of the device after the bend.

In an example of the production of a device with an acute angled proximal opening, which is less than ninety degrees to the longitudinal axis, the laser may form the proximal opening by cutting one end of the tubular body at an acute angle to the longitudinal axis of the device 480. The angle may be determined in order to form a sufficiently large proximal opening of the device. The tubular device may be sized to retain and sufficiently hold a tubular biological structure within its cavity. The non-stretched radius of the device may be determined so as to be small enough to fit and hold the radius of a tubular biological structure. It is advantageous that the proximal opening of the device is relatively large for increased surface area of the connection, increased blood flow and easier joining by a user. If the proximal opening is ninety degrees to the longitudinal axis, it may be restricted to the same dimensions as the tubular part of the device. Cutting the proximal opening to form an acute angled opening facilitates a much larger opening and provides all the advantages of such an opening without affecting the radial dimensions of the tubular part of the device, which are sized to optimally hold a tubular biological structure. The production may be done manually or may be automatic.

The production may include producing a restraining mechanism as shown and described in FIGS. 11a-11e. The stop members may be formed in a similar way to forming the protrusions and may be formed using a laser cut of the body of the tubular member. The stop members may be spaced a distance apart from the plurality of protrusions in order to prevent inadvertent hooking by the stop members of a biological structure being connected with the device. The restraining ring may be made by laser cut of a separate piece of material. The restraining ring may include a net-like structure.

Reference is made to the following examples, which together with the above descriptions illustrate the invention in a non-limiting fashion.

### EXAMPLE 1

### Production of a Connector Device

Twenty rounded filaments made of nitinol were braided into a tubular braid. Each filament had a diameter of about 0.05 mm. Three wider reinforcing filaments were included in the braid. The wider filaments were cut and bent to create hooks at three spaced apart and equidistant positions on the circumference of the braid. The hooks were sized to penetrate a blood vessel wall and an arterial segment on each hook.

### EXAMPLE 2

### Production of a Connector Device

Twenty rounded filaments made of nitinol were braided into a tubular braid. Each filament had a diameter of about 0.05 mm. The tubular braid had a diameter of about 6 mm and a length of about 15 mm. Six wider reinforcing filaments were included in the braid. The wider filaments were cut and bent to create spaced apart hooks in two lines, each line having hooks at three equidistant positions on the circumference of the braid. The first line of hooks was formed about 3 mm from the proximal edge of the device and the second line of hooks was formed about 5 mm from the first line of hooks.

### EXAMPLE 3

### Evaluation of a Nitinol Connector Device for Joining a Severed Blood Vessel to an Artery

A blood vessel was inserted through the device of Example 1. The cut end of the blood vessel was everted over the proximal end of the device. The cut end of the blood vessel was hooked by the three hooks on the external surface of the device. About 5 mm of an arterial segment was stretched over the everted end of the blood vessel and the device. The arterial segment was hooked by the same line of spaced apart hooks on the device, which fixed the blood vessel, resulting in the arterial segment overlying the everted portion of the blood vessel to join the two structures together and form a continuous lumen through the blood vessel and the arterial structure for fluid to flow through.

### EXAMPLE 4

### Evaluation of a Nitinol Connector Device with Two Lines of Hooks for Joining a Severed Blood Vessel to an Artery

A blood vessel was inserted through the device of Example 2. The cut end of the blood vessel was everted over the proximal end of the device. The cut end of the blood vessel was hooked by a first line of three spaced apart hooks on the device. An arterial segment was stretched over the everted end of the blood vessel and the device and hooked by a second line of three spaced apart hooks on the device, resulting in the arterial segment overlying the everted portion of the blood vessel to join the two structures together and form a continuous lumen through the blood vessel and the arterial structure for fluid to flow through.

### EXAMPLE 5

### Production of a Net-Like Connector Device

A hollow tube of nitinol was cut using a laser to form a net-like array of interconnected cells of long sided diamonds. A proximal opening was formed by laser cutting the tube at an acute angle of about fourteen degrees relative to the longitudinal axis of the tube. A plurality of spaced apart protrusions were formed by laser cutting spikes out of the body of the tube to protrude from the external surface of the proximal opening. The production was done automatically.

### EXAMPLE 6

### A Bypass Connection with Two Net-Like Devices

An experiment was conducted on two anesthetized pigs. Blood flow was restricted in the carotid artery using a clamping means. Two openings were made in the carotid artery, one opening before the clamp and the second opening after the clamp. Each opening was about 20 mm. A length of the jugular vein of about 150 mm was removed and used as a graft. A first end of the blood vessel graft was inserted into a first net-like device with an acute angled proximal opening according to an aspect of the present invention as described herein. The device had a proximal opening with a maximal length of about 25 mm and included a complete ring part of the device of about 15 mm. The first end of the graft was everted over the acute angled proximal opening of the first device and fixed with spikes positioned about the proximal opening. The second end of the blood vessel graft was inserted into a second net-like device of the present invention with an acute angled proximal opening and with the same dimensions as the first device. The second end of the blood vessel graft was fixed to the second device in the same way as described for fixing the first end of the graft to the first device. The proximal opening of the first device with the attached first end of the graft was inserted into the first opening of the artery to a superficial depth, such that the tissue surrounding the first opening contacted the spikes on the proximal opening of the first device and was fixed with the spikes to the first device to join the first end of the graft to the first opening in the artery. Similarly, the proximal opening of the second device with the attached second end of the graft was inserted into the second opening of the artery to a superficial depth, such that the tissue surrounding the second opening contacted the spikes on the proximal opening of the second device and was fixed with the spikes to the second device to join the second end of the graft to the second opening in the artery. After the connection, a pulse was detected in the artery and the graft was seen to expand due to blood flowing through the graft. The procedure took only several minutes, significantly less than suturing used in a standard bypass procedure.

One skilled in the art can appreciate from the foregoing description that the broad devices and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

What is claimed is:

1. A device for connecting a biological structure, the device comprising a tubular member that includes:
a longitudinal axis, a radial axis at least substantially perpendicular to the longitudinal axis and a tubular body configured for reversible radial enlargement of the tubular member; a distal opening at a distal end of the tubular member; a proximal opening at a proximal end of the tubular member;
the tubular member including an inner surface and a lumen defining a cavity for receiving and retaining a biological structure; and a plurality of spaced apart protrusions protruding out from the exterior surface of the body of the device for fixing to the device an end of the biological structure everted over the proximal extremity of the device and for fixing an end of a second structure to the external surface of the device to facilitate contact between the end of the second structure and the everted end of the biological structure for connecting the biological structure to the second structure.

2. The device of claim 1, wherein the plurality of spaced apart protrusions are adjacent to the proximal extremity of the tubular member and are positioned and angled to fix the biological structure to the device and the second structure to the device.

3. The device of claim 2, wherein the plurality of spaced apart protrusions comprise a first plurality of protrusions and a second plurality of protrusions, wherein the first plurality of protrusions are configured for fixing the everted biological structure to the device and the second plurality of protrusions are configured for fixing the second structure to the device and for contacting at least part of the second structure with at least the everted part of the biological structure.

4. The device of claim 3, wherein the first plurality of protrusions are configured spaced apart about the circumference of the tubular member in a first at least one line adjacent to the proximal end of the device and the second plurality of protrusions are configured spaced apart about the circumference of the tubular member in a second at least one line adjacent to the proximal end of the device and wherein the first at least one line is nearer to the proximal end of the tubular member than the second at least one line.

5-32. (canceled)

33. A The device of claim 1
wherein the tubular body comprises a net-like design of interconnected cells configured for reversible radial enlargement of the tubular member.

34. (canceled)

35. (canceled)

36. The device of claim 33, wherein the proximal opening is formed at an acute angle to the longitudinal axis of the device.

37. (canceled)

38. The device of claim 33, wherein at least one of the proximal opening and the distal opening is formed at a ninety degrees angle to the longitudinal axis of the device.

39. (canceled)

40. The device of claim 36, wherein the tubular body comprises:
a tubular body first part comprising a right circular hollow cylinder of connected open cells in a net-like design, the first part extending longitudinally from the distal opening until the nearest point of the proximal opening in relation to the distal opening; and
a tubular body second part continuous and in line with the tubular body first part and comprising the same longitudinal axis as the tubular body first part, wherein the tubular body second part comprises an incomplete hollow cylinder, the proximal opening carved out from the cylinder body at an acute angle to the longitudinal axis to form an incomplete ring of connected open cells in a net-like design in a radial line with the proximal opening.

41. The device of claim 40, wherein the maximal length of the proximal opening is greater than the diameter of the tubular body first part.

42. (canceled)

43. The device of claim 41, wherein the proximal opening provides and forms substantially the same angle and size of the proximal opening to an end of a biological structure or non-biological structure affixed to the proximal opening of the device.

44. The device of claim 33, wherein the plurality of spaced apart protrusions comprises a plurality of spaced apart protrusions about the proximal opening of the device and wherein the plurality of spaced apart protrusions are in the same line about the circumference of the proximal opening and wherein at least one protrusion of the plurality of spaced apart protrusions is sized for fixing to the device both a biological structure and a second structure to be joined to the biological structure.

45-51. (canceled)

52. The device of claim 36, wherein the device is configured to connects the biological structure to the second structure at an acute angle equivalent to the angle of the proximal opening of the device.

53. The device of claim 33, further comprising a restraining mechanism.

54. The device of claim 53, wherein the restraining mechanism comprises:
a restraining ring for encircling the device, wherein the encircling restraining ring is freely moveable along the longitudinal axis of the external surface of the device; and
at least one unidirectional stop member fixed on the external surface of the tubular member adjacent to the proximal opening of the device and projecting out from the external surface of the tubular member;
wherein the at least one unidirectional stop member allows the encircling restraining ring to move over the at least one unidirectional stop member in a direction towards the proximal end of the device and wherein the at least one unidirectional stop member prevents the encircling restraining ring from moving back over the at least one unidirectional stop member in a direction towards the distal end of the device.

55. A system for replacement of and/or augmenting suturing in a bypass procedure, the system comprising two devices of claim 1, one device for connecting a first end of a bypass blood vessel to a first opening in a blocked artery on a first side of the blockage and the second device for connecting a second end of the bypass blood vessel to a second opening in the blocked artery on a second side of the blockage.

56. The system of claim 55, wherein the devices comprise a proximal opening at an acute angle to the longitudinal axis of the device and wherein the devices connect the bypass blood vessel to the opening in the artery at an angle corresponding to the angle of the proximal opening of the device.

57. (canceled)

58. The system of claim 55, wherein the devices comprise a proximal opening at a ninety degrees angle to the longitudinal axis of the device and wherein the devices connect the bypass blood vessel to the opening in the artery at a ninety degrees angle.

59. (canceled)

60. A method of joining a biological structure and a second structure, the method comprising:
providing a device of claim 1;
inserting through the distal opening and into the cavity of the device, the biological structure or a part thereof;
stopping insertion of the biological structure when the end of the biological structure protrudes from the proximal end of the tubular member;
everting the protruding end of the biological structure over the proximal opening of the tubular member;
fixing to the device the everted end of the biological structure with the plurality of spaced apart protrusions on the exterior surface of the tubular member, the affixed end adopting the dimensions, shape and angle of the proximal opening; and
manipulating the second structure and the tubular member with the everted and fixed end of the biological structure to facilitate an opening of the second structure to be fixed to the spaced apart protrusions on the external surface of the device and to overlie and be in substantially direct contact with the everted and fixed end of the biological structure.

61-69. (canceled)

70. The device of claim 33,
the tubular body comprising:
a first section, wherein the first section comprises a first longitudinal axis, a radial axis at least substantially perpendicular to the first longitudinal axis, the first longitudinal axis extending from the distal opening until a bend in the tubular body;
a second section, wherein the second section comprises a second longitudinal axis, a radial axis at least substantially perpendicular to the second longitudinal axis, the second longitudinal axis extending from the bend until the proximal opening, wherein the second longitudinal axis is inclined at an angle of less than one hundred and eighty degrees to the first longitudinal axis and the first section and the second section are joined to form a continuous cavity extending from the distal opening until the proximal opening.

71-73. (canceled)

74. The device of claim 1, wherein the tubular member comprises a plurality of interwoven lengths configured for 1) reversible radial enlargement of the tubular member in response to a pushing force being applied to at least one of the first end or the second end of the tubular member, the pushing force acting in the direction of the longitudinal axis, and 2) reversible radial contraction of the tubular member in response to a pulling force being applied to at least one of the first end or the second end of the tubular member, the pulling force acting in the direction of the longitudinal axis.
